(19) United States
(12) Patent Application Publication
(10) Pub. No.: US 2023/0235396 A1
(43) Pub. Date: Jul. 27, 2023

(54) MASS SPECTROMETRY-BASED KIT FOR ERYTHROCYTE BLOOD GROUP GENOTYPING

(71) Applicant: Shanghai Blood Center, Shanghai (CN)

(72) Inventors: Luyi YE, Shanghai (CN); Qixiu YANG, Shanghai (CN); Ziyan ZHU, Shanghai (CN)

(21) Appl. No.: 18/156,775

(22) Filed: Jan. 19, 2023

(30) Foreign Application Priority Data
Jan. 26, 2022 (CN) 202210098021.6

(51) Int. Cl. C12Q 1/6876

(52) U.S. Cl. CPC C12Q 1/6876; C12Q 2600/156

(57) ABSTRACT

The present invention provides a mass spectrometry-based method and kit for erythrocyte blood group genotyping. By taking nucleic acid mass spectrometry as a platform and by designing primer combinations and improving amplification reaction conditions, 61 blood group genetic sites in 21 erythrocyte blood group systems can be simultaneously detected in one reaction, rapid typing of the 21 erythrocyte blood group systems can be realized, and identified phenotypes are all clinically significant erythrocyte antigen phenotypes. The present invention has the characteristics of high sensitivity, strong specificity, simple operation, and rapid and high throughput. The present invention can be applied to difficult blood group identification, blood matching, rare blood group screening, scientific research, routine business development and the like in clinical practice. A

The present application claims the priority of the Chinese application with the application number of 202210098021.6 applied on 2022-01-26, and all the recorded contents serve as a part of the present invention

### REFERENCE TO AN ELECTRONIC SEQUENCE LISTING

The contents of the electronic sequence listing (2023-01-19-SequenceListing.xml; Size: 165,952 bytes; and Date of Creation: Jan. 9, 2023) is herein incorporated by reference in its entirety.

### TECHNICAL FIELD

The present invention relates to the field of biomedicine, in particular to a mass spectrometry-based method and kit for erythrocyte blood group genotyping.

### BACKGROUND

Blood transfusion has a wide range of application in the medical field and is one of the effective life-saving support measures. However, currently, 43 blood group systems and more than 350 blood group antigens have been reported for erythrocytes, but in China, only D antigens of ABO blood group and Rh blood group systems are routinely detected before erythrocyte transfusion. Therefore, if there are incompatible blood group antigens between blood transfusion donors and recipients, it is possible to produce antibodies through iso-immunization, and re-transfusion of incompatible erythrocyte products may cause hemolytic transfusion reaction, etc., and may endanger life in severe cases. Especially for patients who need long-term erythrocyte transfusion, the probability of producing single or various antibodies is further increased, and it is difficult to find compatible blood products. According to statistics, the probability of erythrocyte sensitization caused by single transfusion is about 3%, and this number may be as high as 60% in patients with long-term blood transfusion. From 2013 to 2017, 17% of blood transfusion deaths reported by the US FDA were caused by iso-immunization-induced hemolytic transfusion reaction. In addition, erythrocyte antibodies produced by immunization are also associated with diseases such as hemolytic disease of the newborn. Therefore, clinically significant comprehensive identification of erythrocyte blood group antigens for blood transfusion patients, blood donors, pregnant and lying-in women, etc. is an effective way to solve the problem of difficult blood groups and to improve the efficacy of blood transfusion. Through rapid and high-throughput blood group screening and identification, rare blood group donors can also be screened and reserved in advance to deal with emergencies, etc.

At present, serological methods are routinely used in identification of erythrocyte blood group antigens in China. However, commercial serological detection reagents are not available for most blood group antigens, or the reagents are too expensive to routinely perform serological detection. Therefore, genotyping methods complement and replace the serological methods. Current erythrocyte blood group antigen genotyping kits in China are mainly based on low-throughput technologies, such as PCR-SSP. Some laboratories also use self-built methods for detection, such as PCR-RFLP, sequencing, etc. Internationally, there have been some blood group genotyping products based on medium and high-throughput technologies, such as gene chip technology, suspension array technology, mass spectrometry technology and so on. However, existing medium and high-throughput blood group genotyping products are mainly aimed at Caucasian populations and blacks, etc., and are not suitable for Chinese population due to differences in genetic backgrounds of different populations. At present, in China, there is still a large gap in medium and high-throughput methods and kits for blood group genotyping with independent intellectual property rights. Therefore, in order to improve the accuracy and breadth of blood group antigen genetic diagnosis and improve the detection throughput, it is necessary to develop a high-throughput blood group genotyping method suitable for the Chinese population. A nucleic acid mass spectrometry technology has the characteristics of being accurate, rapid and capable of detecting common mutation types such as SNP and In/Del, and is suitable for genotyping of blood group antigens.

CN110079590A provides a detection method for genotyping of rare erythrocyte antigens, but it can only detect 29 SNP sites, and does not specify blood group antigens, genotypes and phenotypes corresponding to the detected sites, so blood group genotyping that can be used for clinical practice cannot be proved. Blood group genotypes of erythrocytes are very complex. There are many blood groups in different site combinations of different antigens. The same blood group also has multiple genotypes. Therefore, it is necessary to detect more sites simultaneously in order to achieve higher-throughput blood group screening and identification, and it is urgent to find detection methods and products that can detect more erythrocyte blood groups simultaneously.

### SUMMARY OF THE INVENTION

For the problems in the prior art, the present invention provides a mass spectrometry-based method and kit for erythrocyte blood group genotyping. By designing primer combinations and improving amplification reaction conditions, 61 blood group genetic sites in 21 erythrocyte blood group systems can be simultaneously detected in one reaction, rapid typing of the 21 erythrocyte blood group systems can be realized, and identified phenotypes are all clinically significant erythrocyte antigen phenotypes. The present invention has the characteristics of high sensitivity, strong specificity, simple operation, and rapid and high throughput. The present invention can be applied to difficult blood group identification, blood matching, rare blood group screening, scientific research, routine business development and the like in clinical practice.

Blood group genotypes of erythrocytes are very complex. There are many blood groups in different site combinations of different antigens. The same blood group also has multiple genotypes. Therefore, it is necessary to detect more sites simultaneously in order to achieve higher-throughput blood group screening and identification. However, the more sites are detected simultaneously, the low conversion rate of PCR multiplex reaction is likely to occur. Moreover, due to the problems that genes of erythrocyte antigens have genes with very high homology and sequences where some SNP sites are located are rich in GC, etc., and genes where some SNP sites are located have highly homologous sequences, resulting in that when the SNP sites of these genes are detected simultaneously based on mass spectrometry, the situations are prone to occurring that some sites do not have peaks and are not detected, or it is easy to amplify to homologous sequences to generate erroneous results, etc., and there is a problem that it is difficult to detect all sites one time. In the present invention, by screening a large number of primer combinations and adjusting reaction conditions, simultaneous detection of 61 blood group genetic sites in 21 erythrocyte blood group systems can be finally realized, so that rapid typing of the 21 erythrocyte blood group systems can be realized. Moreover, the present invention has high specificity and sensitivity, and rapid and high throughput.

On the one hand, the present invention provides a primer combination for erythrocyte blood group genotyping, including amplification primers and extension primers. The amplification primers include forward primers and reverse primers, and sequences of amplification primer combinations are shown in Table 1. All these amplification primers can be placed in one amplification tube to expand a target gene one time, and a difference of SNP sites can be distinguished one time. The sites distinguished are 61 different SNP sites.

TABLE 1
List of amplification primer combinations for erythrocyte blood group genotyping (for
different SNP sites)

| Blood group | Phenotype (SNP serial | | Forward | Reverse |
|---|---|---|---|---|
| systems | number) | SNP sites | primers | primers |
| Augustine | At(a+)/At(a−) (SNP1) | rs775471940 | SEQ ID NO: 1 | SEQ ID NO: 2 |
| (AUG) | At(a+)/At(a−) (SNP2) | rs45458701 | SEQ ID NO: 4 | SEQ ID NO: 5 |
| | At(a+)/At(a−) (SNP3) | rs759118384 | SEQ ID NO: 7 | SEQ ID NO: 8 |
| Rh (RH) | C/c | rs586178 | SEQ ID NO: 10 | SEQ ID NO: 11 |
| | E/e | rs609320 | SEQ ID NO: 13 | SEQ ID NO: 14 |
| CD59 (CD59) | CD59: +1/CD59: −1 (SNP1) | CD59_c_361delG (see | SEQ ID NO: 16 | SEQ ID NO: 17 |
| | | C. Weinstock, CD59: A | | |
| | | long-known | | |
| | | complement inhibitor | | |
| | | has advanced to a blood | | |
| | | group system) | | |
| | CD59: +1/ CD59: −1 (SNP2) | CD59_c_123delC (see | SEQ ID NO: 19 | SEQ ID NO: 20 |
| | | C. Weinstock, CD59: A | | |
| | | long-known | | |
| | | complement inhibitor | | |
| | | has advanced to a blood | | |
| | | group system) | | |
| Colton (CO) | Co+/Co(a−b−) (SNP1) | rs749625062 | SEQ ID NO: 22 | SEQ ID NO: 23 |
| | Co+/Co(a−b−) (SNP2) | rs777730687 | SEQ ID NO: 25 | SEQ ID NO: 26 |
| | Co^{a}/Co^{b} | rs28362692 | SEQ ID NO: 28 | SEQ ID NO: 29 |
| Cromer | Crom+/Cromerₙᵤₗₗ (SNP1) | rs1131690771 | SEQ ID NO: 31 | SEQ ID NO: 32 |
| (CROM) | Crom+/Cromerₙᵤₗₗ (SNP2) | rs121909603 | SEQ ID NO: 34 | SEQ ID NO: 35 |
| | Crom+/Cromerₙᵤₗₗ (SNP3) | rs762195469 | SEQ ID NO: 37 | SEQ ID NO: 38 |
| | Cr(a+)/Cr(a−) | rs60822373 | SEQ ID NO: 40 | SEQ ID NO: 41 |
| Diego (DI) | Di^{a}/Di^{b} | rs2285644 | SEQ ID NO: 43 | SEQ ID NO: 44 |
| Duffy (FY) | Fy^{a}/Fy^{b} | rs12075 | SEQ ID NO: 46 | SEQ ID NO: 47 |
| Gerbich (GE) | GE+/Leach | GYPC_c_134delC (see | SEQ ID NO: 49 | SEQ ID NO: 50 |
| | | The Blood Group | | |
| | | Antigen FactsBook | | |
| | | (Third Edition) | | |
| | | p506-507) | | |
| | GE+/GEIS+ | GYPC_c_95C_A (see | SEQ ID NO: 52 | SEQ ID NO: 53 |
| | | The Blood Group | | |
| | | Antigen FactsBook | | |
| | | (Third Edition) | | |
| | | p506-507) | | |
| I (I) | I+/I− (SNP1) | rs1177742207 | SEQ ID NO: 55 | SEQ ID NO: 56 |
| | I+/I− (SNP2) | rs56141211 | SEQ ID NO: 58 | SEQ ID NO: 59 |
| | I+/I− (SNP3) | rs755228157 | SEQ ID NO: 61 | SEQ ID NO: 62 |
| | I+/I− (SNP4) | rs774740944 | SEQ ID NO: 64 | SEQ ID NO: 65 |
| | I+/I− (SNP5) | rs201291494 | SEQ ID NO: 67 | SEQ ID NO: 68 |
| | I+/I− (SNP6) | rs55940927 | SEQ ID NO: 70 | SEQ ID NO: 71 |
| Indian (IN) | In^{a}/In^{b} | rs369473842 | SEQ ID NO: 73 | SEQ ID NO: 74 |
| Kidd (JK) | Jk+/Jk(a−b−) (SNP1) | rs538368217 | SEQ ID NO: 76 | SEQ ID NO: 77 |
| | Jk+/Jk(a−b−) (SNP2) | rs78937798 | SEQ ID NO: 79 | SEQ ID NO: 80 |
| | Jk^{a}/Jk^{b} | rs1058396 | SEQ ID NO: 82 | SEQ ID NO: 83 |
| JR (JR) | Jr(a+)/Jr(a−) (SNP1) | rs140207606 | SEQ ID NO: 85 | SEQ ID NO: 86 |
| | Jr(a+)/Jr(a−) (SNP2) | rs548254708 | SEQ ID NO: 88 | SEQ ID NO: 89 |
| | Jr(a+)/Jr(a−) (SNP3) | rs72552713 | SEQ ID NO: 91 | SEQ ID NO: 92 |
| Kell (KEL) | K/k | rs8176058 | SEQ ID NO: 94 | SEQ ID NO: 95 |
| Knops (KN) | Kn^{a}/Kn^{b} | rs41274768 | SEQ ID NO: 97 | SEQ ID NO: 98 |
| LAN (LAN) | Lan+/Lan− (SNP1) | rs769584110 | SEQ ID NO: 100 | SEQ ID NO: 101 |
| | Lan+/Lan− (SNP2) | rs202232534 | SEQ ID NO: 103 | SEQ ID NO: 104 |
| | Lan+/Lan− (SNP3) | rs755723161 | SEQ ID NO: 106 | SEQ ID NO: 107 |
| Lutheran (LU) | Lu+/Lu(a−b−) (SNP1) | KLF1_19-12996560-T- | SEQ ID NO: 109 | SEQ ID NO: 110 |
| | | TG (see | | |
| | | https://gnomad.broadinstitute.org website) | | |
| | Lu+/Lu(a−b−) (SNP2-P1) | rs483352838 | SEQ ID NO: 112 | SEQ ID NO: 113 |
| | Lu+/Lu(a−b−) (SNP2-P2) | rs483352838 | SEQ ID NO: 115 | SEQ ID NO: 116 |
| | Lu^{a}/Lu^{b} | rs28399653 | SEQ ID NO: 118 | SEQ ID NO: 119 |
| P1PK (P1PK) | P^{k}+/p (SNP1) | rs1398859071 | SEQ ID NO: 121 | SEQ ID NO: 122 |
| | P^{k}+/p (SNP2) | rs387906280 | SEQ ID NO: 124 | SEQ ID NO: 125 |
| | P^{k}+/p (SNP3) | A4GALT_c_418 (see | SEQ ID NO: 127 | SEQ ID NO: 128 |
| | | Y.-C. Wang, Functional | | |
| | | chaaracterisation of a | | |
| | | complex mutation in | | |
| | | the | | |
| | | α(1,4)galactosyltransferase | | |
| | | gene in Taiwanese | | |
| | | individuals with p | | |
| | | phenotype) | | |
| | P^{k}+/p (SNP4) | A4GALT_MG812384 | SEQ ID NO: 130 | SEQ ID NO: 131 |
| | | (see GenBank | | |
| | | Homo sapiens truncated | | |
| | | alpha | | |
| | | 1,4-galactosyltransferase | | |
| | | gene, complete cds | | |
| | | GenBank: | | |
| | | MG812384.1) | | |
| | P^{k}+/p (SNP5) | rs1189809232 | SEQ ID NO: 133 | SEQ ID NO: 134 |
| | P^{k}+/p (SNP6) | rs755279796 | SEQ ID NO: 136 | SEQ ID NO: 137 |
| | P^{k}+/p (SNP7) | rs778387354 | SEQ ID NO: 139 | SEQ ID NO: 140 |
| H(H) | H+/Para-Bombay (SNP1) | rs777455020 | SEQ ID NO: 142 | SEQ ID NO: 143 |
| | H+/Para-Bombay (SNP2) | rs573412368 | SEQ ID NO: 145 | SEQ ID NO: 146 |
| | H+/Para-Bombay (SNP3) | rs574691621 | SEQ ID NO: 148 | SEQ ID NO: 149 |
| MNS (MNS) | S/s | rs7683365 | SEQ ID NO: 151 | SEQ ID NO: 152 |
| Vel (VEL) | Vel+/Vel− (SNP1) | rs1169340827 | SEQ ID NO: 154 | SEQ ID NO: 155 |
| | Vel+/Vel− (SNP2) | rs554492306 | SEQ ID NO: 157 | SEQ ID NO: 158 |
| | Vel+/Vel− (SNP3) | rs566629828 | SEQ ID NO: 160 | SEQ ID NO: 161 |
| | Vel+/Vel− (SNP4) | rs899095555 | SEQ ID NO: 163 | SEQ ID NO: 164 |
| | Vel+/Vel− (SNP5) | rs1182690110 | SEQ ID NO: 166 | SEQ ID NO: 167 |
| | Vel+/Vel− (SNP6) | rs1207554936 | SEQ ID NO: 169 | SEQ ID NO: 170 |
| Yt (YT) | Yt+/Yt(a−b−) (SNP1) | rs772244054 | SEQ ID NO: 172 | SEQ ID NO: 173 |
| | Yt+/Yt(a−b−) (SNP2) | rs114782198 | SEQ ID NO: 175 | SEQ ID NO: 176 |
| | Yt+/Yt(a−b−) (SNP3) | rs771039143 | SEQ ID NO: 178 | SEQ ID NO: 179 |
| | Yt+/Yt(a−b−) (SNP4) | ACHE (see | SEQ ID NO: 181 | SEQ ID NO: 182 |
| | | https://gnomad.broadinstitute.org website) | | |
| | Yt/Yt | rs1799805 | SEQ ID NO: 184 | SEQ ID NO: 185 |

Notes:
1) The names of the blood group systems are named according to the International Society of Blood Transfusion (ISBT), and system names (system symbols) are listed in Table of blood group systems v. 10.0.
2) In the phenotype, “blood group system symbol +”, for example, Co+, I+, etc., indicates that a protein corresponding to a gene encoded by the blood group system is in a wild type.
3) For the expression of other phenotypes and blood group antigens in the phenotype, refer to The Blood Group Antigen FactsBook (Third Edition).

Further, the above primer combination is characterized in that sequences of extension primer combinations are shown in Table 2.

TABLE 2
List of extension primer combinations for erythrocyte blood group genotyping

| Blood group | Phenotype (SNP serial | | |
|---|---|---|---|
| systems | number) | SNP sites | Extension primers |
| Augustine | At(a+)/At(a−) (SNP1) | rs775471940 | SEQ ID NO: 3 |
| (AUG) | At(a+)/At(a−) (SNP2) | rs45458701 | SEQ ID NO: 6 |
| | At(a+)/At(a−) (SNP3) | rs759118384 | SEQ ID NO: 9 |
| Rh (RH) | C/c | rs586178 | SEQ ID NO: 12 |
| | E/e | rs609320 | SEQ ID NO: 15 |
| CD59 (CD59) | CD59: +1/CD59: −1 (SNP1) | CD59_c_361delG | SEQ ID NO: 18 |
| | CD59: +1/ CD59: −1 (SNP2) | CD59_c_123delC | SEQ ID NO: 21 |
| Colton (CO) | Co+/Co(a−b−) (SNP1) | rs749625062 | SEQ ID NO: 24 |
| | Co+/Co(a−b−) (SNP2) | rs777730687 | SEQ ID NO: 27 |
| | Co^{a}/Co^{b} | rs28362692 | SEQ ID NO: 30 |
| Cromer | Crom+/Cromerₙᵤₗₗ (SNP1) | rs1131690771 | SEQ ID NO: 33 |
| (CROM) | Crom+/Cromerₙᵤₗₗ (SNP2) | rs121909603 | SEQ ID NO: 36 |
| | Crom+/Cromerₙᵤₗₗ (SNP3) | rs762195469 | SEQ ID NO: 39 |
| | Cr(a+)/Cr(a−) | rs60822373 | SEQ ID NO: 42 |
| Diego (DI) | Di^{a}/Di^{b} | rs2285644 | SEQ ID NO: 45 |
| Duffy (FY) | Fy^{a}/Fy^{b} | rs12075 | SEQ ID NO: 48 |
| Gerbich (GE) | GE+/Leach | GYPC_c_134delC | SEQ ID NO: 51 |
| | GE+/GEIS+ | GYPC_c_95C_A | SEQ ID NO: 54 |
| I (I) | I+/I− (SNP1) | rs1177742207 | SEQ ID NO: 57 |
| | I+/I− (SNP2) | rs56141211 | SEQ ID NO: 60 |
| | I+/I− (SNP3) | rs755228157 | SEQ ID NO: 63 |
| | I+/I− (SNP4) | rs774740944 | SEQ ID NO: 66 |
| | I+/I− (SNP5) | rs201291494 | SEQ ID NO: 69 |
| | I+/I− (SNP6) | rs55940927 | SEQ ID NO: 72 |
| Indian (IN) | In^{a}/In^{b} | rs369473842 | SEQ ID NO: 75 |
| Kidd (JK) | Jk+/Jk(a−b−) (SNP1) | rs538368217 | SEQ ID NO: 78 |
| | Jk+/Jk(a−b−) (SNP2) | rs78937798 | SEQ ID NO: 81 |
| | Jk^{a}/Jk^{b} | rs1058396 | SEQ ID NO: 84 |
| JR (JR) | Jr(a+)/Jr(a−) (SNP1) | rs140207606 | SEQ ID NO: 87 |
| | Jr(a+)/Jr(a−) (SNP2) | rs548254708 | SEQ ID NO: 90 |
| | Jr(a+)/Jr(a−) (SNP3) | rs72552713 | SEQ ID NO: 93 |
| Kell (KEL) | K/k | rs8176058 | SEQ ID NO: 96 |
| Knops (KN) | Kn^{a}/Kn^{b} | rs41274768 | SEQ ID NO: 99 |
| LAN (LAN) | Lan+/Lan− (SNP1) | rs769584110 | SEQ ID NO: 102 |
| | Lan+/Lan− (SNP2) | rs202232534 | SEQ ID NO: 105 |
| | Lan+/Lan− (SNP3) | rs755723161 | SEQ ID NO: 108 |
| Lutheran (LU) | Lu+/Lu(a−b−) (SNP1) | KLF1_19-12996560- | SEQ ID NO: 111 |
| | | T-TG | |
| | Lu+/Lu(a−b−) (SNP2-P1) | rs483352838 | SEQ ID NO: 114 |
| | Lu+/Lu(a−b−) (SNP2-P2) | rs483352838 | SEQ ID NO: 117 |
| | Lu^{a}/Lu^{b} | rs28399653 | SEQ ID NO: 120 |
| P1PK (P1PK) | P^{k}+/p (SNP1) | rs1398859071 | SEQ ID NO: 123 |
| | P^{k}+/p (SNP2) | rs387906280 | SEQ ID NO: 126 |
| | P^{k}+/p (SNP3) | A4GALTc418 | SEQ ID NO: 129 |
| | P^{k}+/p (SNP4) | A4GALT_MG812384 | SEQ ID NO: 132 |
| | P^{k}+/p (SNP5) | rs1189809232 | SEQ ID NO: 135 |
| | P^{k}+/p (SNP6) | rs755279796 | SEQ ID NO: 138 |
| | P^{k}+/p (SNP7) | rs778387354 | SEQ ID NO: 141 |
| H(H) | H+/Para-Bombay (SNP1) | rs777455020 | SEQ ID NO: 144 |
| | H+/Para-Bombay (SNP2) | rs573412368 | SEQ ID NO: 147 |
| | H+/Para-Bombay (SNP3) | rs574691621 | SEQ ID NO: 150 |
| MNS (MNS) | S/s | rs7683365 | SEQ ID NO: 153 |
| Vel (VEL) | Vel+/Vel− (SNP1) | rs1169340827 | SEQ ID NO: 156 |
| | Vel+/Vel− (SNP2) | rs554492306 | SEQ ID NO: 159 |
| | Vel+/Vel− (SNP3) | rs566629828 | SEQ ID NO: 162 |
| | Vel+/Vel− (SNP4) | rs899095555 | SEQ ID NO: 165 |
| | Vel+/Vel− (SNP5) | rs1182690110 | SEQ ID NO: 168 |
| | Vel+/Vel− (SNP6) | rs1207554936 | SEQ ID NO: 171 |
| Yt(YT) | Yt+/Yt(a−b−) (SNP1) | rs772244054 | SEQ ID NO: 174 |
| | Yt+/Yt(a−b−) (SNP2) | rs114782198 | SEQ ID NO: 177 |
| | Yt+/Yt(a−b−) (SNP3) | rs771039143 | SEQ ID NO: 180 |
| | Yt+/Yt(a−b−) (SNP4) | ACHE | SEQ ID NO: 183 |
| | Yt^{a}/Yt^{b} | rs1799805 | SEQ ID NO: 186 |

Blood group genotype and phenotype information of the 61 blood group genetic sites of the present invention are shown in Table 3 and Table 4, wherein for sequences in Table 4, sequences in parentheses are polymorphic sites.

TABLE 3
Blood group genotype information of 61 blood group genetic sites

| Blood | | | | | | |
|---|---|---|---|---|---|---|
| group | Phenotype (SNP | | Poly- | | | |
| systems | serial number) | SNP sites | morphism | Genotype→phenotype | | |
| Augustine | At(a+)/At(a-) | rs775471940 | AGTCC | ins/ins→At(a+) | del/del→At(a-) | ins/del→At(a+) |
| (AUG) | (SNP1) | | AGCAT | | | |
| | | | >- | | | |
| | At(a+)/At(a-) | rs45458701 | G>A,C | GG→At(a+) | AA→At(a-) | GA→At(a+) |
| | (SNP2) | | | | | |
| | At(a+)/At(a-) | rs759118384 | AGG>- | ins/ins→At(a+) | del/del→At(a-) | ins/del→At(a+) |
| | (SNP3) | | | | | |
| Rh (RH) | C/c | rs586178 | G>A,C | GG→C+c- | CC→C-c+ | GC→C+c+ |
| | E/e | rs609320 | C>A,G, | CC→E-e+ | GG→E+e- | GC→E+e+ |
| | | | T | | | |
| CD59 | CD59:+1/ | CD59_c_361delG | G>- | ins/ins→CD59: | del/del→CD59: | ins/del→CD59: |
| (CD59) | CD59:-1 (SNP1) | (see C. Weinstock, | | +1 | -1 | +1 |
| | | CD59: A long-known | | | | |
| | | complement inhibitor | | | | |
| | | has advanced to a | | | | |
| | | blood group system) | | | | |
| | CD59:+1/ | CD59_c_123delC (see | C>- | ins/ins→CD59: | del/del→CD59: | ins/del→CD59: |
| | CD59:-1 (SNP2) | C. Weinstock, CD59: | | +1 | -1 | +1 |
| | | A long-known | | | | |
| | | complement inhibitor | | | | |
| | | has advanced to a | | | | |
| | | blood group system) | | | | |
| Colton | Co+/Co(a-b-) | rs749625062 | CT>- | ins/ins→Co+ | del/del→Co | ins/del→Co+ |
| (CO) | (SNP1) | | | | (a-b-) | |
| | Co+/Co(a-b-) | rs777730687 | C>- | ins/ins→Co+ | del/del→Co | ins/del→Co+ |
| | (SNP2) | | | | (a-b-) | |
| | Coª/Co^{b} | rs28362692 | C>A,T | CC→Co(a+b-) | TT→Co(a-b+) | CT→Co(a+b+) |
| Cromer | Crom+/Cromerₙᵤₗₗ | rs1131690771 | C>A | CC→Crom+ | AA→Cromerₙᵤₗₗ | CA→Crom+ |
| (CROM) | (SNP1) | | | | | |
| | Crom+/Cromerₙᵤₗₗ | rs121909603 | G>A | GG→Crom+ | AA→Cromerₙᵤₗₗ | GA→Crom+ |
| | (SNP2) | | | | | |
| | Crom+/Cromerₙᵤₗₗ | rs762195469 | C>T | CC→Crom+ | TT→Cromerₙᵤₗₗ | CT→Crom+ |
| | (SNP3) | | | | | |
| | Cr(a+)/Cr(a-) | rs60822373 | G>C,T | GG→Cr(a+) | CC→Cr(a-) | GC→Cr(a+) |
| Diego (DI) | Diª/Di^{b} | rs2285644 | G>A,T | GG→Di(a-b+) | AA→Di(a+b-) | GA→Di(a+b+) |
| Duffy (FY) | Fyª/Fy^{b} | rs12075 | G>A | GG→Fy(a+b-) | AA→Fy(a-b+) | GA→Fy(a+b+) |
| Gerbich | GE+/Leach | GYPC_c_134delC | C>- | ins/ins→GE+ | del/del→Leach | ins/del→GE+ |
| (GE) | | (see The Blood Group | | | | |
| | | Antigen FactsBook | | | | |
| | | (Third Edition) | | | | |
| | | p506-507) | | | | |
| | GE+/GEIS+ | GYPC_c_95C_A (see | C>A | CC→GE+ | AA→GEIS+ | CA→GE+ |
| | | The Blood Group | | | | |
| | | Antigen FactsBook | | | | |
| | | (Third Edition) | | | | |
| | | p506-507) | | | | |
| I (I) | I+/I- (SNP1) | rs1177742207 | G>A | GG→I+ | AA→I- | GA→I+ |
| | I+/I- (SNP2) | rs56141211 | G>A | GG→I+ | AA→I- | GA→I+ |
| | I+/I- (SNP3) | rs755228157 | G>A | GG→I+ | AA→I- | GA→I+ |
| | I+/I- (SNP4) | rs774740944 | G>A | GG→I+ | AA→I- | GA→I+ |
| | I+/I- (SNP5) | rs201291494 | T>A | TT→I+ | AA→I- | TA→I+ |
| | I+/I- (SNP6) | rs55940927 | G>A | GG→I+ | AA→I- | GA→I+ |
| Indian (IN) | Inª/In^{b} | rs369473842 | G>A,C | GG→In(a-b+) | CC→In(a+b-) | GC→In(a+b+) |
| Kidd (JK) | Jk+/Jk(a-b-) | rs538368217 | G>A | GG>Jk+ | AA→Jk(a-b-) | GA→Jk+ |
| | (SNP1) | | | | | |
| | Jk+/Jk(a-b-) | rs78937798 | G>A | GG>Jk+ | AA→Jk(a-b-) | GA→Jk+ |
| | (SNP2) | | | | | |
| | Jk^{a}/Jk^{b} | rs1058396 | G>A,C, | GG→Jk(a+b-) | AA→Jk(a-b+) | GA→Jk(a+b+) |
| | | | T | | | |
| JR (JR) | Jr(a+)/Jr(a-) | rs140207606 | G>A,T | GG→Jr(a+) | AA→Jr(a-) | GA→Jr(a+) |
| | (SNP1) | | | | | |
| | Jr(a+)/Jr(a-) | rs548254708 | G>A,T | GG→Jr(a+) | AA→Jr(a-) | GA→Jr(a+) |
| | (SNP2) | | | | | |
| | Jr(a+)/Jr(a-) | rs72552713 | G>A | GG→Jr(a+) | AA→Jr(a-) | GA→Jr(a+) |
| | (SNP3) | | | | | |
| Kell (KEL) | K/k | rs8176058 | G>A,C | GG→K-k+ | AA→K+k- | GA→K+k+ |
| Knops (KN) | Knª/Kn^{b} | rs41274768 | G>A | GG→Kn(a+b-) | AA→Kn(a-b+) | GA→Kn(a+b+) |
| LAN | Lan+/Lan- | rs769584110 | C>- | ins/ins→Lan+ | del/del→Lan- | ins/del→Lan+ |
| (LAN) | (SNP1) | | | | | |
| | Lan+/Lan- | rs202232534 | G>A,T | GG→Lan+ | TT→Lan | GT→Lan+ |
| | (SNP2) | | | | weak | |
| | Lan+/Lan- | rs755723161 | G>- | ins/ins→Lan+ | del/del→Lan- | ins/del→Lan+ |
| | (SNP3) | | | | | |
| Lutheran | Lu+/Lu(a-b-) | KLF1_19-12996560-T- | →C | del/del→Lu+ | ins/ins→ND | del/ins→Lu |
| (LU) | (SNP1) | TG (see https:// | | | | (a-b-) |
| | | gnomad.broadinstitute. | | | | |
| | | org website) | | | | |
| | Lu+/Lu(a-b-) | rs483352838 | →GCCG | del/del→Lu+ | ins/ins→ND | del/ins→Lu |
| | (SNP2-P1) | | GGC | | | (a-b-) |
| | Luª/Lu^{b} | rs28399653 | G>A,C | GG→Lu(a-b+) | AA→Lu(a+b-) | GA→Lu(a+b+) |
| P1PK | p^{k}+/p (SNP1) | rs1398859071 | A>- | ins/ins→p^{k}+ | del/del→p | ins/del→p^{k}+ |
| (P1PK) | p^{k}+/p (SNP2) | rs387906280 | ->G | del/de1→^{p}+ | ins/ins→p | del/ins→p^{k}+ |
| | p^{k}+/p (SNP3) | A4GALT_c_418 (see | CAGAT | InsShort/ | InsLong/ | InsShort/InsLong→ |
| | | Y. -C. Wang, | GCTCC | InsShort→p^{k}+ | InsLong→p | pₖ+ |
| | | Functional | C>TGG | | | |
| | | chaaracterisation of a | ACCTG | | | |
| | | complex mutation in | CTGGA | | | |
| | | the | CCTGC | | | |
| | | α(1,4)galactosyl- | TGGAC | | | |
| | | transferase gene in | CTGCT | | | |
| | | Taiwanese individuals | GGAAC | | | |
| | | with p phenotype) | A | | | |
| | p^{k}+/p (SNP4) | A4GALT_MG812384 | →CACA | del/del→p^{k}+ | ins/ins→p | del/ins→p^{k}+ |
| | | (see GenBank | CCC | | | |
| | | Homo sapiens | | | | |
| | | truncated alpha | | | | |
| | | 1,4-galactosyl- | | | | |
| | | transferase gene, | | | | |
| | | complete cds | | | | |
| | | GenBank: MG812384.1) | | | | |
| | p^{k}+/p (SNP5) | rs1189809232 | C>- | ins/ins→p^{k}+ | del/del→p | ins/del→p^{k}+ |
| | p^{k}+/p (SNP6) | rs755279796 | T>A | TT→p^{k}+ | AA→p | TA→p^{k}+ |
| | p^{k}+/p (SNP7) | rs778387354 | ACGTG | ins/ins→p+ | del/del→p | ins/del→p^{k}+ |
| | | | GCCTC | | | |
| | | | GAACC | | | |
| | | | GCGTG | | | |
| | | | CCCTG | | | |
| | | | G>- | | | |
| H (H) | H+/Para-Bombay | rs777455020 | AA>- | ins/ins→H+ | del/del→Para- | ins/del→H+ |
| | (SNP1) | | | | Bombay | |
| | H+/Para-Bombay | rs573412368 | CT>- | ins/ins→H+ | del/del→Para- | ins/del→H+ |
| | (SNP2) | | | | Bombay | |
| | H+/Para-Bombay | rs574691621 | G>A,T | GG→H+ | AA→Para-Bombay | GA→H+ |
| | (SNP3) | | | | | |
| MNS | S/s | rs7683365 | G>A,C, | AA→S+s- | GG→S-s+ | AG→S+s+ |
| (MNS) | | | T | | | |
| Vel (VEL) | Vel+/Vel- (SNP1) | rs1169340827 | G>A,T | GG→Vel+ | AA→Vel- | GA→Vel+ |
| | Vel+/Vel- (SNP2) | rs554492306 | G>A | GG >Vel+ | AA→NA | GA→Vel+ |
| | Vel+/Vel- (SNP3) | rs566629828 | AGCCT | ins/ins→Vel+ | del/del→Vel- | ins/del→Vel+ |
| | | | AGGGG | | | |
| | | | CTGTG | | | |
| | | | TC>- | | | |
| | Vel+/Vel- (SNP4) | rs899095555 | C>T | CC→Vel+ | TT→NA | CT→Vel+ |
| | Vel+/Vel- (SNP5) | rs1182690110 | T>A,G | TT→Vel+ | AA,GG,AG→ | TA,TG→Vel+ |
| | | | | | Vel | |
| | | | | | weak/Vel- | |
| | Vel+/Vel- (SNP6) | rs1207554936 | CAGCA | ins/ins→Vel+ | del/del→Vel- | ins/del→Vel+ |
| | | | TGC>- | | | |
| Yt (YT) | Yt+/Yt(a-b-) | rs772244054 | GAG>- | ins/ins→Yt+ | del/del→Yt(a | ins/del→Yt+ |
| | (SNP1) | | | | -b-) | |
| | Yt+/Yt(a-b-) | rs114782198 | G>A | GG→Yt+ | AA→NA | GA→Yt+ |
| | (SNP2) | | | | | |
| | Yt+/Yt(a-b-) | rs771039143 | →C | del/del→Yt+ | ins/ins→Yt(a-b-) | del/ins→Yt+ |
| | (SNP3) | | | | | |
| | Yt+/Yt(a-b-) | ACHE (see https:// | TTGCT | ins/ins→Yt+ | del/del→Yt(a-b-) | ins/del→Yt+ |
| | (SNP4) | gnomad.broadinstitute. | C>C | | | |
| | | org website) | | | | |
| | Yt^{a}/Yt^{b} | rs1799805 | G>T | GG→Yt(a+b-) | TT→Yt(a-b+) | GT→Yt(a+b+) |

Notes:
1. Some gene polymorphisms are not listed in genotype to phenotype correspondence because no reports about the corresponding phenotypes of the gene polymorphisms have been retrieved.
2. ND, not detected; NA, no information available.

TABLE 4
Sequences before and after SNP sites of 61 blood group genetic sites

| | Phenotype | | |
|---|---|---|---|
| Blood | (SNP | | |
| group | serial | SNP | Sequences before and after SNP sites([ ]with |
| systems | number) | sites | underline is the SNP site) |
| Augustine | At(a+)/ | rs775471940 | GACTCTACCCCTTCTATCTAGATCTCAGTCCTGGCTTTC |
| (AUG) | At(a−) | | TCTGTCTGCTTCATCTTCACTATCACCATTGGGATGTTT |
| | (SNP1) | | CCAGCCGTGACTGTTGAGGTCA[AGTCCAGCAT/-]CGC |
| | | | AGGCAGCAGCACCTGGGGTGAGGATGCCACAGGTTTC |
| | | | CAGGATGGGAACAGACAGGATCTTGAGTTGGGCTGGA |
| | | | AGTGGGGAAGGGAGGGAGCCTGG |
| | At(a+)/ | rs45458701 | CAGCCGCTGGCTGCCAAGCCTGGTGCTGGCCCGGCTG |
| | At(a−) | | GTGTTTGTGCCACTGCTGCTGCTGTGCAACATTAAGCC |
| | (SNP2) | | CCGCCGCTACCTGACTGTGGTCTTC[G/A/C]AGCACGAT |
| | | | GCCTGGTTCATCTTCTTCATGGCTGCCTTTGCCTTCTCC |
| | | | AACGGCTACCTCGCCAGCCTCTGCATGTGCTTCGGGCC |
| | | | CAAGTGAGTAGGGCT |
| | At(a+)/ | rs759118384 | CACTGGCCTGTTCTGTCAGCCCTGCCCCCTCTCCCCCT |
| | At(a−) | | AAGAGCCTGAGGAGGCCTATGGCAGGGCCAAGACAG |
| | (SNP3) | | GGCCTCACACTGTTCCTGCCCCCAGC[AGG/-]CCCCTG |
| | | | AGGGAGGGAGCTGTCAGCCAGGGAAAACCGAGAACA |
| | | | CCATCACCATGACAACCAGTCACCAGCCTCAGGACAG |
| | | | GTAAGGGGTAAGGGGCTGGGC |
| Rh(RH) | C/c | rs586178 | CTTGATAGGATGCCACGAGCCCCTTTTGATCCTCTAAG |
| | | | GAAGCGTCATAGTGGGTAAAAAAATAGAAGAGGAGAA |
| | | | TGAGAGCTGCTTCCAGTGTTAGGGC[G/A/C]CAGAGGG |
| | | | GCAGGCAGCGCCGGACAGACCGCGGGTACTTAGAGCT |
| | | | CATCCTGTGTCCGTCTCTGTGCAGGGGTTCCACCAGCA |
| | | | CCAGGCATCACCCCTCTC |
| | E/e | rs609320 | GCCAAGGATGACCCTGAGATGGCTGTCACCACACTGA |
| | | | CTGCTAGAGCATAGTAGGTGTTGAACATGGCATTCTTC |
| | | | CTTTGGATTGGACTTCTCAGCAGAG[C/A/G/T]AGAGTT |
| | | | GACACTTGGCCAGAACATCCACAAGAAGAGGGCGCCT |
| | | | GGGGGCCAGAGAGGGTGGTTGGCCAGAATCACACTCC |
| | | | TGCTCCAAAGGTCTGAGCCT |
| CD59 | CD59: +1/ | CD59_c_ | CAAGTGTATAACAAGTGTTGGAAGTTTGAGCATTGCAA |
| (CD59) | CD59: −1 | 361delG | TTTCAACGACGTCACAACCCGCTTGAGGGAAAATGAG |
| | (SNP1) | | CTAACGTACTACTGCTGCAAGAAGGACCTGTGTAACTT |
| | | | TAACGAACAGCTTGAAAATGGTGGGACATCCTTATCAG |
| | | | AGAAAACAGTTCTTCTGCTGGTGACTCCATTTCTG[G/-] |
| | | | CAGCAGCCTGGAGCCTTCATCCCTAAGTCAACACCAG |
| | | | GAGAGCTTCTCCCAAACTCCCCGTTCCTGCGTAGTCCG |
| | | | CTTTCTCTTGCTGCCACATTCTAAAGGCTTGATATTTTC |
| | | | CAAATGGATCCTGTTGGGAAAGAATAAAATTAGCTTGA |
| | | | GCAACCTGGCTAAGATAGAGG |
| | CD59: +1/ | CD59_c_ | ACATTTGTGCGGGAGTGGAAGTATACCACAAGTTGCTG |
| | CD59: −1 | 123delC | ACTTTGGGCCCATATTAATGGAGATGGTGGGCTGCCAG |
| | (SNP2) | | GGGACAAGTCAGTGCTGCTTTAAGAGATCCTGACTTTC |
| | | | TTCCTGATTCTAGGTCATAGCCTGCAGTGCTACAACTGT |
| | | | CCTAACCCAACTGCTGACTGCAAAACAGC[C/-]GTCAA |
| | | | TTGTTCATCTGATTTTGATGCGTGTCTCATTACCAAAGC |
| | | | TGGTAAGAGCCTCCCCTGTCTGTCTCCTAAATGTAATG |
| | | | GGGTAATAAGTGCCTGGGAAAAAAATTGTGCCACTGTA |
| | | | ATCCTCATTAGGCTTGCATCAAGTAAT |
| Colton | Co+/Co(a− | rs749625062 | GGCAGCGGTCTCAGGCCAAGCCCCCTGCCAGCATGGC |
| (CO) | b−) | | CAGCGAGTTCAAGAAGAAGCTCTTCTGGAGGGCAGTG |
| | (SNP1) | | GTGGCCGAGTTCCTGGCCACGACCCT[CT/-]TTGTCTTC |
| | | | ATCAGCATCGGTTCTGCCCTGGGCTTCAAATACCCGGT |
| | | | GGGGAACAACCAGACGGCGGTCCAGGACAACGTGAA |
| | | | GGTGTCGCTGGCCTTCGG |
| | Co+/Co(a− | rs777730687 | GCTGACCTCAGGGTGAGTGCAGGGCCCTGGGTGACAT |
| | b−) | | GTGTGTGCCTCTCTCCTCCTTCCCAGACACCACCCAAA |
| | (SNP2) | | CCATCTCTGAGGACACAGACAACGA[C/-]CTTGTCCCA |
| | | | CCCCTCGAGCTCTGCATCAGAACCAAGTGAGTCAAGC |
| | | | CCCTCTCTGGCTTTGAGCCTCACCCTGAATAGCTTTGA |
| | | | GGAGCCATGGTTGGGG |
| | Co^{a}/Co^{b} | rs28362692 | AGGGCAGTGGTGGCCGAGTTCCTGGCCACGACCCTCT |
| | | | TTGTCTTCATCAGCATCGGTTCTGCCCTGGGCTTCAAAT |
| | | | ACCCGGTGGGGAACAACCAGACGG[C/A/T]GGTCCAGG |
| | | | ACAACGTGAAGGTGTCGCTGGCCTTCGGGCTGAGCAT |
| | | | CGCCACGCTGGCGCAGAGTGTGGGCCACATCAGCGGC |
| | | | GCCCACCTCAACCCGGCT |
| Cromer | Crom+/ | rs1131690771 | TTTCCCGAGGATACTGTAATAACGTACAAATGTGAAGA |
| (CROM) | Cromerₙᵤₗₗ | | AAGCTTTGTGAAAATTCCTGGCGAGAAGGACTCAGTG |
| | (SNP1) | | ATCTGCCTTAAGGGCAGTCAATGGT[C/A]AGATATTGAA |
| | | | GAGTTCTGCAATCGTAAGTTCTTCATCTTTTTAGAAAA |
| | | | GTTCTGGGAATGGAATGTATCTTAAATTTATTTTTATATA |
| | | | CCTTTGGAGTGA |
| | Crom+/ | rs121909603 | GTTTTCCCGAGGATACTGTAATAACGTACAAATGTGAA |
| | Cromerₙᵤₗₗ | | GAAAGCTTTGTGAAAATTCCTGGCGAGAAGGACTCAG |
| | (SNP2) | | TGATCTGCCTTAAGGGCAGTCAATG[G/A]TCAGATATTG |
| | | | AAGAGTTCTGCAATCGTAAGTTCTTCATCTTTTTAGAA |
| | | | AAGTTCTGGGAATGGAATGTATCTTAAATTTATTTTTATA |
| | | | TACCTTTGGAGT |
| | Crom+/Cr | rs762195469 | CACATAGTTACCTTCTTTGTGTGTATGCCTGATAATTTAA |
| | Cromerₙᵤₗₗ | | TTTTAAAAAATCAATTTGTATTCTATTCTAGAGAAATCA |
| | (SNP3) | | TGCCCTAATCCGGGAGAAATA[C/T]GAAATGGTCAGAT |
| | | | TGATGTACCAGGTGGCATATTATTTGGTGCAACCATCTC |
| | | | CTTCTCATGTAACACAGGGTAAGTTTGGGCATACTAAA |
| | | | ACCCTGTATT |
| | Cr(a+)/ | rs60822373 | TATAATCTTTAACATGTTTTGATCTTATTTGTAAAAATAC |
| | Cr(a−) | | TTTACTAGTTTTATTTATTTAAAAGATGTTGGAATTGTTT |
| | | | TTTAAGAAATTTATTGTCCA[G/C/T]CACCACCACAAAT |
| | | | TGACAATGGAATAATTCAAGGGGAACGTGACCATTATG |
| | | | GATATAGACAGTCTGTAACGTATGCATGTAATAAAGGAT |
| | | | TCACCATGAT |
| Diego | Di^{a}/Di^{b} | rs2285644 | ACTCACACACTGAAGCTCCACGTTCCTGAAGATGAGC |
| (DI) | | | GGCAGCAGGACGCGCCGCAGCGGCACAGTGAGGATG |
| | | | AGGACGAAGGGCAGGGCCAGGGAGGCC[G/A/T]GCGT |
| | | | GGACTTCACCACCCACAGCACTGCCAGGCAGATGATC |
| | | | TGGATGCCCGTGAATAAGTGCATGCGCCAGGTCTTCAC |
| | | | CTGCAGGCGGAGGCTGGGGTC |
| Duffy | Fy^{a}/Fy^{b} | rs12075 | GAGCTCTCCCCCTCAACTGAGAACTCAAGTCAGCTGG |
| (FY) | | | ACTTCGAAGATGTATGGAATTCTTCCTATGGTGTGAATG |
| | | | ATTCCTTCCCAGATGGAGACTATG[G/A]TGCCAACCTG |
| | | | GAAGCAGCTGCCCCCTGCCACTCCTGTAACCTGCTGGA |
| | | | TGACTCTGCACTGCCCTTCTTCATCCTCACCAGTGTCCT |
| | | | GGGTATCCTAGCT |
| Gerbich | GE+/Leach | GYPC_c_ | AGCTTGGGCCAAGGTGCTGCTAGGCATGGAGAATCTTC |
| (GE) | | 134delC | CTCTCTGACCTCAGATTCTTGTCCTCTGTTCACAGAGC |
| | | | CTGATCCAGGGATGTCTGGATGGC[C/-]GGATGGCAGAA |
| | | | TGGAGACCTCCACCCCCACCATAATGGACATTGTCGTC |
| | | | ATTGCAGGTGAGCTCTCATCACAGAGCCCTTCAAGCAG |
| | | | CCAGGGTGGGGGG |
| | GE+/GEIS+ | GYPC_c_95C_ | GCTAGGCATGGAGAGTCTTCCTCTCTGACCTCAGATTC |
| | | A | TTGTCCTCTGTTCACAGAGCCTGATCCGGGGATGGCCT |
| | | | CTGCCTCCACCACAATGCATACTA[C/A]CACCATTGCAG |
| | | | GTGAGTTCTCATCACAGAGCCTCACCATAATGGAAACT |
| | | | GCCGTGACTTCAGATGAGCTCTCATCACAGAGCCCTTT |
| | | | AAGCAGCCAGGGT |
| I(I) | I+/I− | rs1177742207 | CATGACTCTCATCTCTACGCTTCTTCTTTATCAACATTG |
| | (SNP1) | | CAGGTGTTCCTGGCTCTATGCCAAATGCATCCTGGACT |
| | | | GGAAACCTCAGAGCTATAAAGTG[G/A]AGTGACATGGA |
| | | | AGACAGACACGGAGGCTGCCACGGTGAGGCTCTCGTT |
| | | | CCATGCTTCTAGGCCACTGCCTGTTGGTGTTAGCAGGA |
| | | | AGGTAGCTGTGGAA |
| | I+/I− | rs56141211 | ATTCTGTAAGTTCATCACCCTTTTGAAAGCAAGCATGT |
| | (SNP2) | | TTTGACTCTGTTTCTTGTTCTTTCTTTTGCAGGCCACTA |
| | | | TGTACATGGTATTTGTATCTATG[G/A]AAACGGAGACTT |
| | | | AAAGTGGCTGGTTAATTCACCAAGCCTGTTTGCTAACA |
| | | | AGTTTGAGCTTAATACCTACCCCCTTACTGTGGAATGCC |
| | | | TAGAACTGAGG |
| | I+/I− | rs755228157 | TTCCCCCTGAAAACCAACCGGGAGATAGTTCAGCATCT |
| | (SNP3) | | GAAAGGATTTAAAGGGAAAAATATCACCCCAGGGGTG |
| | | | CTGCCTCCTGACCATGCAATTAAGC[G/A]AACTAAATAT |
| | | | GTCCACCAAGAGCATACAGATAAAGGTGGCTTTTTTGT |
| | | | GAAAAATACTAATATTTTGAAAACTTCACCTCCACATC |
| | | | AGCTGACCATCTAC |
| | I+/I− | rs774740944 | CTTCTTTATCAACATTGCAGGTGTTCCTGGCTCTATGCC |
| | (SNP4) | | AAATGCATCCTGGACTGGAAACCTCAGAGCTATAAAGT |
| | | | GGAGTGACATGGAAGACAGACAC[G/A]GAGGCTGCCA |
| | | | CGGTGAGGCTCTCGTTCCATGCTTCTAGGCCACTGCCT |
| | | | GTTGGTGTTAGCAGGAAGGTAGCTGTGGAATCCAGGG |
| | | | TTCTCATGGAGAGAG |
| | I+/I− | rs201291494 | CCTGTAATCACGCCTTAGAGAAAATGCCAGTCTTTTTG |
| | (SNP5) | | TGGGAAAATATATTACCATCACCTTTGCGAAGTGTCCCT |
| | | | TGCAAGGATTACCTGACCCAGAA[T/A]CACTACATCAC |
| | | | AAGTCCCCTGTCGGAAGAAGAGGCTGCATTCCCTTTG |
| | | | GCCTATGTCATGGTCATCCATAAGGACTTTGACACCTTT |
| | | | GAAAGGCTCTTTA |
| | I+/I− | rs55940927 | GGAGACTTAAAGTGGCTGGTTAATTCACCAAGCCTGTT |
| | (SNP6) | | TGCTAACAAGTTTGAGCTTAATACCTACCCCCTTACTGT |
| | | | GGAATGCCTAGAACTGAGGCATC[G/A]CGAAAGAACC |
| | | | CTCAATCAGAGTGAAACTGCGATACAACCCAGCTGGTA |
| | | | TTTTTGAGCTATTCATGAGCTACTCATGACTGAAGGGA |
| | | | AACTGCAGCTGGGA |
| Indian | In^{a}/In^{b} | rs369473842 | AAGAATCTAACATTTCTATTTCTTCCCATAGATTTGAATA |
| (IN) | | | TAACCTGCCGCTTTGCAGGTGTATTCCACGTGGAGAAA |
| | | | AATGGTCGCTACAGCATCTCTC[G/A/C]GACGGAGGCC |
| | | | GCTGACCTCTGCAAGGCTTTCAATAGCACCTTGCCCAC |
| | | | AATGGCCCAGATGGAGAAAGCTCTGAGCATCGGATTTG |
| | | | AGACCTGCAGGTAA |
| Kidd (JK) | Jk+/Jk(a− | rs538368217 | CCTCCTGTCTTAACAGGACTCAGTCTTTCAGCCCCATT |
| | b−) | | TGAGGACATCTACTTTGGACTCTGGGGTTTCAACAGCT |
| | (SNP1) | | CTCTGGCCTGCATTGCAATGGGAG[G/A]AATGTTCATG |
| | | | GCGCTCACCTGGCAAACCCACCTCCTGGCTCTTGGCTG |
| | | | TGGTGAGTCTCCCACGCCCCTGGGGGAGGGCTGCTCA |
| | | | TGACTACAGGATCTC |
| | Jk+/Jk(a− | rs78937798 | TTTTAATATGAATATGATCTGGAAGTTACTAGTGTTATTT |
| | b−) | | ATGTGCAAGTGCAACCAAAGCTCACCCAGGAAATGTC |
| | (SNP2) | | CGTGCTGTGTCTCTTGCCCCACA[G/A]GTCATTAATAGC |
| | | | ATCTGGGCTCTATGGCTACAATGCCACCCTGGTGGGAG |
| | | | TACTCATGGCTGTCTTTTCGGACAAGGGAGACTATTTC |
| | | | TGGTGGCTGTTA |
| | Jk^{a}/Jk^{b} | rs1058396 | TCAATCCCACCCTCAGTTTCCTTCCAGAACATCCTGCC |
| | | | TTTAGTCCTGAGTTCTGACCCCTCCTGTCTTAACAGGA |
| | | | CTCAGTCTTTCAGCCCCATTTGAG[G/A/C/T]ACATCTAC |
| | | | TTTGGACTCTGGGGTTTCAACAGCTCTCTGGCCTGCAT |
| | | | TGCAATGGGAGGAATGTTCATGGCGCTCACCTGGCAA |
| | | | ACCCACCTCCTGGCTCT |
| JR (JR) | Jr(a+)/Jr | rs140207606 | GGCCCGTGGAACATAAGTCTTCCTGAGGCCAATAAGGT |
| | (a−) | | GAGGCTATCAAACAACTTGAAGATGGAATATCGAGGCT |
| | (SNP1) | | GATGAATGGAGAAGATGATTGTTC[G/A/T]TCCCTGCTT |
| | | | AGACATCCTAAGTTAAAAGTGAGACAATACTAAGTCAT |
| | | | TAAATATCTGAAACTTGTATTTCTCAGTAAAATACTCTA |
| | | | TTCTTGCCTTTAGA |
| | Jr(a+)/Jr | rs548254708 | TGGTCAGGGAAATGAGATGAGGACACTTGATTTCCATT |
| | (a−) | | GTTCCTAGCTTGGGAATGCAGTCACAGTGACAGACAA |
| | (SNP2) | | GGAAGACATACCGTAAATCCATATC[G/A/T]TGGAATGC |
| | | | TGAAGTACTGAAGCCATGACAGCCAAGATGCAATGGT |
| | | | TGTGAGATTGACCAACAGACCTGAAAAAATCTACAAA |
| | | | AAGCAAATACTAAAAGTC |
| | Jr(a+)/Jr | rs72552713 | TGTCACATAATCAACTGGAAGCACATTGAACTATCAGC |
| | (a−) | | CAAAGCACTTACCCATATAGAAACAGAGGAAACAGAA |
| | (SNP3) | | AATGCAAACCCACTAATACTTACTT[G/A]TACCACGTAA |
| | | | CCTGAATTACATTTGAAATTGGCAGGTCGCGGTGCTCC |
| | | | ATTTATCAGAACATCTCCAGATAATCCACTTGGATCTTT |
| | | | CCTTGCAGCTAAG |
| Kell | K/k | rs8176058 | CCTCACCTGGATGACTGGTGTGTGTGGAGAGGCAGGA |
| (KEL) | | | TGAGGTCCTAGGTAGGCTCTGAAGAAAGGGAAATGGC |
| | | | CATACTGACTCATCAGAAGTCTCAGC[G/A/C]TTCGGTT |
| | | | AAAGTTTAAGGAAGTCCATTTACCAGAGATGCGCCAG |
| | | | CCTCCAAGCTTTAAAGGAGAGAGAGGGGGCTGAGCAT |
| | | | AAGGATCCGTGGAGCCCAT |
| Knops | Kn^{a}/Kn^{b} | rs41274768 | TGGAGACTTCTACAGCAACAATAGAACATCTTTTCACA |
| (KN) | | | ATGGAACGGTGGTAACTTACCAGTGCCACACTGGACC |
| | | | AGATGGAGAACAGCTGTTTGAGCTT[G/A]TGGGAGAA |
| | | | CGGTCAATATATTGCACCAGCAAAGATGATCAAGTTGG |
| | | | TGTTTGGAGCAGCCCTCCCCCTCGGTGTATTTCTACTAA |
| | | | TAAATGCACAGCTCC |
| LAN | Lan+/Lan− | rs769584110 | TCTCTGAGTAGCCAGGAAATAATAATGTGCCCTGGACA |
| (LAN) | (SNP1) | | GGTGAGGGCCAGGGCTCAGATTACCTGTAGTAGGTGC |
| | | | CAAACCAATTGAGGGGCATGTACAG[C/-]TGGATAATGT |
| | | | AGGTGCCAAAGAGCACATAGTCCCCAACCTGTGGCAA |
| | | | TCAAGGAAGCAGAGCATGTCACGGGGGGCCTGCAGGC |
| | | | CGCTCTTCTTGTGTCA |
| | Lan+/Lan− | rs202232534 | CAAGACACCAGGGCCAAGTTCTCAGCTGCAAACGCCA |
| | (SNP2) | | CAGTCCAGAGGAGCAGGAGACCAGGGCTGTGCCTGA |
| | | | ACTTGATCCAGATGCCCATTGCCAGAC[G/A/T]CTGCCG |
| | | | TGCCTGGCTCCGCTCCACGACAAGCAGCCACAGGCCA |
| | | | CAGGCGCCGGCCAGACTCTCCAGCACGGAGGCCAGA |
| | | | AGTAGATAGCTTGGCAGTGGG |
| | Lan+/Lan− | rs755723161 | AGTCCCTCACCTGCTGGCCCAAGTCTGCCCTTGCCCAC |
| | (SNP3) | | CACCACTGTGGGCTGTTCCAAGACACCAGGGCCAAGT |
| | | | TCTCAGCTGCAAACGCCACAGTCCA[G/-]AGGAGCAGG |
| | | | AGACCAGGGCTGTGCCTGAACTTGATCCAGATGCCCAT |
| | | | TGCCAGACGCTGCCGTGCCTGGCTCCGCTCCACGACA |
| | | | AGCAGCCACAGGCCAC |
| Lutheran | Lu+/Lu(a− | KLF1_19- | TTCGGAGGATCACTCGGGTTGGGTGCGCCCTGCCCTGC |
| (LU) | b−) | 12996560-T- | GAGCCCGGGCTCCCGACGCCTTCGTGGGCCCAGCCCT |
| | (SNP1) | TG | GGCTCCAGCCCCGGCCCCCGAGCCC[-/C]AAGGCGCTG |
| | | | GCGCTGCAACCGGTGTACCCGGGGCCCGGCGCCGGCT |
| | | | CCTCGGGTGGCTACTTCCCGCGGACCGGGCTTTCAGTG |
| | | | CCTGCGGCGTCGGGCG |
| | Lu+/Lu(a− | rs483352838 | CCGGGTACATCGCGGGGTACCCGGACAGTAGCCCGTA |
| | b−) | | GGGGGCGCCCGACGCCGCAGGCACTGAAAGCCCGGT |
| | (SNP2-P1) | | CCGCGGGAAGTAGCCACCCGAGGAGCC[-/GCCGGGC] |
| | | | CCCGGGTACACCGGTTGCAGCGCCAGCGCCTTGGGCT |
| | | | CGGGGGCCGGGGCTGGAGCCAGGGCTGGGCCCACGA |
| | | | AGGCGTCGGGAGCCCGGGCTCGCAGGG |
| | Lu^{a}/Lu^{b} | rs28399653 | GAGAAAGGACCCAGAGAGAGAGAGACTGAGGAGCGC |
| | | | TGGGACACCCGGAGCTGAGAGCCTGCCCCGCGCCCAC |
| | | | AGACCGACCGCTCGGGAGCTCGCCCCC[G/A/C]CCTAG |
| | | | CCTCGGCTGAGATGCAGGGCTCTGAGCTCCAGGTCAC |
| | | | AATGCACGACACCCGGGGCCGCAGTCCCCCATACCAG |
| | | | CTGGACTCCCAGGGGCGCCTG |
| P1PK | P^{k}+/p | rs1398859071 | GGACAGCATAGGTGGCACTGAGCAGCCGCGGCAGCTC |
| (P1PK) | (SNP1) | | CTCGGGGTTGATGTCCTCAAAGTACTTCTTCCAGTCCT |
| | | | GCCAGGGGATGGGGTAGAAGGCCTC[A/-]GGGGGCAGG |
| | | | GTGGTGACGCCGCGGCAGGCGCGGCTCTCGGCCAGGC |
| | | | TGCGGATGGAACACCACTTCTTGAAGACCCGCGTGAG |
| | | | CAGCTGCGGGCCCTGGT |
| | P^{k}+/p | rs387906280 | GCCTCCCCGGGAAGGGCGGCCCAGTGCCCCATCAGGA |
| | (SNP2) | | GCAGGTTGGGGAGGTGACCTGGCGGGCCCCTCACAAG |
| | | | TACATTTTCATGGCCTCGTGCGTCGT[-/G]CAGTAGCGG |
| | | | GCATGCAGCTGGGCCAGCAGTGCCCTGGACGTGGCCT |
| | | | CGAACCGCGTGCCCTGGCTCTTCTTGTTCCACACGTGG |
| | | | ACAGCATAGGTGGCAC |
| | P^{k}+/p | A4GALT_c__ | CGAATCCCACGTGCTGGTCCTGATGAAAGGGCTTCCGG |
| | (SNP3) | 418 | GTGGCAACGCCTCTCTGCCCCGGCACCTGGGCATCTCA |
| | | | CTTCTGAGCTGCTTCCCGAATGTC[CAGATGCTCCC/T |
| | | | GGACCTGCTGGACCTGCTGGACCTGCTGGAACA]G |
| | | | CTGGACCTGCGGGAGCTGTTCCGGGACACACCCCTGG |
| | | | CCGACTGGTACGCGGCCGTGCAGGGGCGCTGGGAGCC |
| | | | CTACCTGCTGCCCGTGCTCTCCGAC |
| | P^{k}+/p | A4GALT_MG | GTGGCAACGCCTCTCTGCCCCGGCACCTGGGCATCTCA |
| | (SNP4) | 812384 | CTTCTGAGCTGCTTCCCGAATGTCCAGATGCTCCCGCT |
| | | | GGACCTGCGGGAGCTGTTCCGGGA[-/CACACCC]CACA |
| | | | CCCCTGGCCGACTGGTACGCGGCCGTGCAGGGGCGCT |
| | | | GGGAGCCCTACCTGCTGCCCGTGCTCTCCGACGCCTCC |
| | | | AGGATCGCACTCATGTGGAAG |
| | P^{k}+/p | rs1189809232 | CTCAGAAGTGAGATGCCCAGGTGCCGGGGCAGAGAGG |
| | (SNP5) | | CGTTGCCACCCGGAAGCCCTTTCATCAGGACCAGCAC |
| | | | GTGGGATTCGGGGTGAGTTCTGGCGG[C/-]CGACTCCA |
| | | | CCGAGCACATGAACAGGAAGTTGGGGTTGGTCCGGTC |
| | | | TGAAGTCTCCAGGAAGAAGATGTTGCCTGGAGTGGGG |
| | | | CCGTGGGAGGGTGGGGTG |
| | P^{k}+/p | rs755279796 | TCCCGCAGGTCCAGCGGGAGCATCTGGACATTCGGGA |
| | (SNP6) | | AGCAGCTCAGAAGTGAGATGCCCAGGTGCCGGGGCAG |
| | | | AGAGGCGTTGCCACCCGGAAGCCCTT[T/A]CATCAGGA |
| | | | CCAGCACGTGGGATTCGGGGTGAGTTCTGGCGGCCGA |
| | | | CTCCACCGAGCACATGAACAGGAAGTTGGGGTTGGTC |
| | | | CGGTCTGAAGTCTCCAGG |
| | P^{k}+/p | rs778387354 | GTTGGGGAGGTGACCTGGCGGGCCCCTCACAAGTACA |
| | (SNP7) | | TTTTCATGGCCTCGTGCGTCGTGGGGCAGTAGCGGGCA |
| | | | TGCAGCTGGGCCAGCAGTGCCCTGG[ACGTGGCCTCG |
| | | | AACCGCGTGCCCTGG/-]CTCTTCTTGTTCCACACGTG |
| | | | GACAGCATAGGTGGCACTGAGCAGCCGCGGCAGCTCC |
| | | | TCGGGGTTGATGTCCTCAAAGTACTTCTTCCAGTCCTG |
| | | | CCAGG |
| H(H) | H+/Para- | rs777455020 | GTTGGCCAGGTAGACAGTGTCTCCGCCAGCCAGGTAG |
| | Bombay | | GCAGCCCAGAAGCCGAAGGTGCCAATGGTCATAATGG |
| | (SNP1) | | TGTGGTTGCACTGTGTGAGCAGGGCA[AA/-]GTCTTTCC |
| | | | ACGGTGTAGCCTCCTGTCCATCGCCAGCAAACGTCACA |
| | | | TCGCCCTGGGAGGTGTCGATGTTTTCTTTACACCACTC |
| | | | CATGCCGTTGCTGGTG |
| | H+/Para- | rs573412368 | GCCGACAAAGGTGCGCGGGCGGTCCCCTGTGCGGCCC |
| | Bombay | | AGGCGGAGCTGACCCAGCACACTCTGCGCCTCTTCCC |
| | (SNP2) | | GAAGGTGGTCGTGCAGGGTGAACTCT[CT/-]GCGGATC |
| | | | TGTTCCCGGAGATGGTGGAAGAAAGTCCAAGAGCAGG |
| | | | GGAAGCCAGAGAGCTTCAGGAAAGGATCTCTCAAGTC |
| | | | CGCGTACTCCTCCGACATC |
| | H+/Para- | rs574691621 | TGCCGTGCCCGGAACCAGTCCATGGCCTGCCGGAGGT |
| | Bombay | | AGGCGCTGTCGCCCACCACACCCTTCCAGCGCTGAGG |
| | (SNP3) | | CATAACCTGCAGATAGTCCCCACGGC[G/A/T]CACGTGG |
| | | | ACGCCGACAAAGGTGCGCGGGCGGTCCCCTGTGCGGC |
| | | | CCAGGCGGAGCTGACCCAGCACACTCTGCGCCTCTTC |
| | | | CCGAAGGTGGTCGTGCAGG |
| MNS | S/s | rs7683365 | GCATTTGAAACAAGCAATGGATAGTTTAAAATGGAATG |
| (MNS) | | | ACTTTTATTCTTTGTCAAATATTAACATACCTGGTACAG |
| | | | TGAAACGATGGACAAGTTGTCCC[G/A/C/T]TTTCTCCTA |
| | | | TAAAGCAAAATTTCAATGTAAGTCCAAATAAGAAAGAC |
| | | | ATGTGCAAAGAAAAAAATCATTTTGGAATCAAACTGTT |
| | | | CTGCGGGTTTCCTTT |
| Vel | Vel+/Vel− | rs1169340827 | CTGGCCACCTGTCTTGATCTCCCCACCGAGAAGGCCCC |
| (VEL) | (SNP1) | | GCCCCTCCCGCTGCAGCCCCACAGCATGCAGCCCCAG |
| | | | GAGAGCCACGTCCACTATAGTAGGT[G/A/T]GGAGGAC |
| | | | GGCAGCAGGGACGGAGTCAGCCTAGGGGCTGTGTCCA |
| | | | GCACAGAAGAGGCCTCACGCTGCCGCAGGTGAGGGG |
| | | | CCTGAGGGCAGCCTGCCAGC |
| | Vel+/Vel− | rs554492306 | ACAGTGAAGCCACAGCCTGGCCACCTGTCTTGATCTCC |
| | (SNP2) | | CCACCGAGAAGGCCCCGCCCCTCCCGCTGCAGCCCCA |
| | | | CAGCATGCAGCCCCAGGAGAGCCAC[G/A]TCCACTATA |
| | | | GTAGGTGGGAGGACGGCAGCAGGGACGGAGTCAGCC |
| | | | TAGGGGCTGTGTCCAGCACAGAAGAGGCCTCACGCTG |
| | | | CCGCAGGTGAGGGGCCTG |
| | Vel+/Vel− | rs566629828 | CGAGAAGGCCCCGCCCCTCCCGCTGCAGCCCCACAGC |
| | (SNP3) | | ATGCAGCCCCAGGAGAGCCACGTCCACTATAGTAGGTG |
| | | | GGAGGACGGCAGCAGGGACGGAGTC[AGCCTAGGGG |
| | | | CTGTGTC/-]CAGCACAGAAGAGGCCTCACGCTGCCGC |
| | | | AGGTGAGGGGCCTGAGGGCAGCCTGCCAGCCATAGCA |
| | | | GGCTGGTGTCTCCCTCCAGAGACGCCTGCCCTAAC |
| | Vel+/Vel− | rs899095555 | GCCCCAGGAGAGCCACGTCCACTATAGTAGGTGGGAG |
| | (SNP4) | | GACGGCAGCAGGGACGGAGTCAGCCTAGGGGCTGTGT |
| | | | CCAGCACAGAAGAGGCCTCACGCTGC[C/T]GCAGGTG |
| | | | AGGGGCCTGAGGGCAGCCTGCCAGCCATAGCAGGCTG |
| | | | GTGTCTCCCTCCAGAGACGCCTGCCCTAACCCCTGCTA |
| | | | CCGGCCCCATCACCCTCC |
| | Vel+/Vel− | rs1182690110 | TAGGGGGCCCCTCATGCGGCCCTGGCCTGGGGCTCAC |
| | (SNP5) | | CTCCAGTTGGTTCTCACCCCAGGATCTCCCAGAGGCTG |
| | | | TGCACGGGCAAGCTGGGCATCGCCA[T/A/G]GAAGGTG |
| | | | CTGGGCGGCGTGGCCCTCTTCTGGATCATCTTCATCCT |
| | | | GGGCTACCTCACAGGCTACTATGTGCACAAGTGCAAAT |
| | | | AAATGCTGCCCCGCATG |
| | Vel+/Vel− | rs1207554936 | CTGCCGCCCTCCATCCGCTTGTTTTACAGTGAAGCCAC |
| | (SNP6) | | AGCCTGGCCACCTGTCTTGATCTCCCCACCGAGAAGG |
| | | | CCCCGCCCCTCCCGCTGCAGCCCCA[CAGCATGC/-]AG |
| | | | CCCCAGGAGAGCCACGTCCACTATAGTAGGTGGGAGG |
| | | | ACGGCAGCAGGGACGGAGTCAGCCTAGGGGCTGTGTC |
| | | | CAGCACAGAAGAGGCCTCACGCTG |
| Yt (YT) | Yt+/Yt(a− | rs772244054 | CCCTTCCCCACGGTCCGACCACTCATTAGAGGAGGGG |
| | b−) | | CCCCTGTGGCCGTAGGGGAAGAGGCCGTGTTCACAGC |
| | (SNP1) | | CGCCGGAGGTGGGAGAGGAAGAGGAG[GAG/-]AAGCT |
| | | | GGTGGAGGAGGAGGAGGGGCAGGGGGAGGCCGGGCC |
| | | | TCGGAGCAGCCTCCCCATGGGTGAAGCCTGGGCAGGT |
| | | | GCTGGGAGCCTCCGAGGCTAGG |
| | Yt+/Yt(a− | rs114782198 | CCCTGACCAAGGCTGCTTGGGCTCCGGTGGCAGAAAG |
| | b−) | | CGACGGGGTCCCATGGGTGGCTCCGCAAAGGGGATGC |
| | (SNP2) | | CCAGGAAAGCAGAGACAGGGCCCCCG[G/A]GGGTCTT |
| | | | CAGGCGAATGCCCCGCAGCCGGCCCCCACGCACCGTC |
| | | | ACCAGCAGCTCTGCATCCTCCCGGCCCTCAGCCCCCAC |
| | | | TCCTCCACCCAGGAGCCA |
| | Yt+/Yt(a− | rs771039143 | GTGGGAGAGGAAGAGGAGGAGAAGCTGGTGGAGGAG |
| | b−) | | GAGGAGGGGCAGGGGGAGGCCGGGCCTCGGAGCAGC |
| | (SNP3) | | CTCCCCATGGGTGAAGCCTGGGCAGGTG[-/C]TGGGAG |
| | | | CCTCCGAGGCTAGGGGGAGAAGAGAGGGGTTACACCT |
| | | | GGCGGGCTCCCACTCCCCTCCTCCCAGCCGCTGCCCGC |
| | | | TGGCCCCTGCATACCGGTG |
| | Yt+/Yt(a− | ACHE | GGGGGCTCAGCAGTACGTTAGTCTGGACCTGCGGCCG |
| | b−) | | CTGGAGGTGCGGCGGGGGCTGCGCGCCCAGGCCTGCG |
| | (SNP4) | | CCTTCTGGAACCGCTTCCTCCCCAAA[TTGCTC/C]AGC |
| | | | GCCACCGGTATGCAGGGGCCAGCGGGCAGCGGCTGGG |
| | | | AGGAGGGGAGTGGGAGCCCGCCAGGTGTAACCCCTCT |
| | | | CTTCTCCCCCTAGCCTCGGAGGC |
| | Yt^{a}/Yt^{b} | rsl799805 | AGAAGCCCTCATGCCTGGGTCCCTGCAGGGAGGGGAG |
| | | | GGACCGTTGGGACCCAGAGGAGCCAGCTTCACGCCAG |
| | | | CTAGCCACTAGTTACCTGCAGGCCGT[G/T]GAAGTCTC |
| | | | CCGCGTTGATGAGGGCCTCTGGGGTGTCACTGAGGAA |
| | | | GTCTCCATCTACCACAGGCACGAAGGAGAACCGGAAG |
| | | | ACGCTTTCTTGAGGCAGC |

On another hand, the present invention provides a kit for genotyping, including primer combinations shown in Table 1 and Table 2.

On yet another hand, the present invention provides a mass spectrometry chip for genotyping, including primer combinations shown in Table 1 and Table 2.

On yet another hand, the present invention provides a method for genotyping by mass spectrometry detection, including the following steps:

(1) by using an amplification primer mix in primer combinations as claimed in claim 1, amplifying genes to be detected by multiplex PCR;

(2) purifying an amplification product obtained in Step (1) by an alkaline phosphatase;

(3) by using an extension primer mix in the primer combinations as claimed in claim 1, extending and amplifying a purified product in Step (2) by a single base; and

(4) conducting sample application on a single-base extended product obtained in Step (3) onto a chip for mass spectrometry detection.

Further, during multiplex PCR reaction in Step (1), a concentration of the amplification primer mix used is 0.04 to 0.4 μM (final concentration).

Further, during multiplex PCR reaction in Step (1), an amplification reaction system used is shown in Table 5.

TABLE 5
Multiplex PCR amplification reaction system

| Components | Volume (μL) |
|---|---|
| Water, HPLC grade | 0.8 |
| 10 × PCR Buffer with 20 mM MgCl₂ | 0.5 |
| 25 mM MgCl₂ | 0.4 |
| 25 mM dNTP Mix | 0.1 |
| 0.2-2.0 uM Primer Mix | 1 |
| 5 U/μl PCR Enzyme | 0.2 |
| 5-20 ng/μL DNA | 2 |
| Total volume | 5 |

Further, during multiplex PCR reaction in Step (1), cycle conditions of amplification reaction are as follows: 95° C., 2 minutes; 45 cycles: 95° C., 30 seconds, 56° C., 30 seconds, 72° C., 60 seconds, 72° C., 5 minutes; keeping a temperature of 4° C.

Further, the alkaline phosphatase in Step (2) is a shrimp alkaline phosphatase, and a premixed solution system for purification treatment with the alkaline phosphatase in Step (2) is shown in

TABLE 6
SAP premixed solution system

| Components | Volume (μL) |
|---|---|
| Nanopure Water, Autoclaved | 1.53 |
| SAP Buffer | 0.17 |
| SAP Enzyme (1.7 U/ul) | 0.30 |
| Total volume | 2 |

Further, a single-base extension and amplification system in Step (3) is shown in Table 7.

TABLE 7
Single-base extension premixed solution system

| Components | Volume (μL) |
|---|---|
| Nanopure Water, Autoclaved | 0.619 |
| iPLEX Buffer | 0.200 |
| iPLEX Termination Mix | 0.200 |
| Extend Primer Mix | 0.94 |
| iPLEX Enzyme | 0.041 |
| Total volume | 2 |

On yet another hand, the present invention provides the primer combinations shown in Table 1 and Table 2 or the above kit or the above mass spectrometry chip for use in simultaneous detection of genotyping detection of 61 SNP sites of erythrocyte blood groups.

A mass spectrometry-based method and kit for erythrocyte blood group genotyping provided by the present invention have the following beneficial effects.

1. 61 Blood group genetic sites in 21 erythrocyte blood group systems can be simultaneously detected in two reactions.

2. Rapid typing of the 21 erythrocyte blood group systems is realized, and identified phenotypes are all clinically significant erythrocyte antigen phenotypes.

3. High sensitivity, strong specificity, simple operation, and rapid and high throughput are realized.

4. The present invention can be applied to difficult blood group identification, blood matching, rare blood group screening, scientific research, routine business development and the like in clinical practice.

### BRIEF DESCRIPTION OF DRAWINGS

FIG. 1 is a representative detection mass spectrogram provided by Embodiment 1;

FIG. 2 is a detection mass spectrogram of an rs548254708 site provided by Embodiment 1;

FIG. 3 is a detection mass spectrogram of an rs676785 site by using initial amplification primers provided by Embodiment 2;

FIG. 4 is a detection mass spectrogram of amplifying homozygous GG→C+c− by amplification primers after the rs676785 site is replaced provided by Embodiment 2;

FIG. 5 is a detection mass spectrogram of amplifying heterozygous GC→C+c+by amplification primers after the rs676785 site is replaced provided by Embodiment 2;

FIG. 6 is a detection mass spectrogram of amplifying homozygous CC→C−c+by amplification primers after the rs676785 site is replaced provided by Embodiment 2;

FIG. 7 is a detection mass spectrogram of KLF1_19-12996560-T-TG before replacement with rs586178 provided by Embodiment 3;

FIG. 8 is a detection mass spectrogram of KLF1_19-12996560-T-TG after replacement with rs586178 provided by Embodiment 3;

FIG. 9 is a detection mass spectrogram of KLF1_19-12996560-T-TG after replacement with rs586178 at a corresponding amplification primer concentration adjusted to 3 times the concentration provided by Embodiment 3;

FIG. 10 is a detection mass spectrogram of rs483352838 before replacement with rs586178 provided by Embodiment 3;

FIG. 11 is a detection mass spectrogram of rs483352838 after replacement with rs586178 provided by Embodiment 3;

FIG. 12 is a detection mass spectrogram of rs483352838 after replacement with rs586178 at an amplification primer concentration adjusted to 2 times the concentration provided by Embodiment 3;

FIG. 13 is a detection mass spectrogram of rs7683365 before amplification primers are replaced provided by Embodiment 4;

FIG. 14 is a detection mass spectrogram of rs7683365 after the amplification primers are replaced provided by Embodiment 4;

FIG. 15 is a detection mass spectrogram of rs778387354 before amplification primers are replaced provided by Embodiment 5;

FIG. 16 is a detection mass spectrogram of rs778387354 after the amplification primers are replaced provided by Embodiment 5;

FIG. 17 is a detection mass spectrogram obtained during single-base extension of an extension primer rs483352838-v1 provided by Embodiment 6;

FIG. 18 is a detection mass spectrogram obtained during single-base extension of an extension primer rs483352838-v2 provided by Embodiment 6;

FIG. 19 is a detection mass spectrogram obtained during single-base extension of an extension primer rs483352838-v3 provided by Embodiment 6; and

FIG. 20 is a detection mass spectrogram obtained during single-base extension of an extension primer rs483352838-v4 provided by Embodiment 6.

### DETAILED DESCRIPTION OF EMBODIMENTS

The present invention will be further described in detail below in combination with embodiments. It should be pointed out that the following embodiments are intended to facilitate the understanding of the present invention, but do not have any limiting effect on it. Reagents used in the embodiments are all known products, and are obtained by purchasing commercially available products.

### Embodiment 1 Methods and Steps for Erythrocyte Blood Group Gene Detection

In this embodiment, 155 cases of blood gene DNAs are used for simultaneously detecting 61 blood group genetic sites in 21 erythrocyte blood group systems, so as to perform rapid blood group genotyping.

Genotyping detection of this embodiment includes the following steps.

1. Sample Preparation:

Genes (DNA) of 155 cases of blood samples are extracted, and concentrations thereof are normalized to 5 to 20 ng/μL for subsequent detection experiments.

2. Primer Design

In this embodiment, PCR amplification primers and single-base extension probes are designed for clinically significant blood group antigens, especially difficult blood groups that may appear in clinical practice in China, in combination with their genetic backgrounds in the Chinese population. Primer sequences are shown in Table 8.

TABLE 8
List of primer combinations

| Blood group | Phenotype (SNP serial | | | Reverse | Extension |
|---|---|---|---|---|---|
| systems | number) | SNP sites | Forward primers | primers | primers |
| Augustine | At(a+)/At(a−) (SNP1) | rs775471940 | SEQ ID NO: 1 | SEQ ID NO: 2 | SEQ ID NO: 3 |
| (AUG) | At(a+)/At(a−) (SNP2) | rs45458701 | SEQ ID NO: 4 | SEQ ID NO: 5 | SEQ ID NO: 6 |
| | At(a+)/At(a−) (SNP3) | rs759118384 | SEQ ID NO: 7 | SEQ ID NO: 8 | SEQ ID NO: 9 |
| Rh(RH) | C/c | rs586178 | SEQ ID NO: 10 | SEQ ID NO: 11 | SEQ ID NO: 12 |
| | E/e | rs609320 | SEQ ID NO: 13 | SEQ ID NO: 14 | SEQ ID NO: 15 |
| CD59 (CD59) | CD59: +1/CD59: −1 | CD59_c_361delG | SEQ ID NO: 16 | SEQ ID NO: 17 | SEQ ID NO: 18 |
| | (SNP1) | | | | |
| | CD59: +1/CD59: −1 | CD59_c_123delC | SEQ ID NO: 19 | SEQ ID NO: 20 | SEQ ID NO: 21 |
| | (SNP2) | | | | |
| Colton (CO) | Co+/Co(a−b−) (SNP1) | rs749625062 | SEQ ID NO: 22 | SEQ ID NO: 23 | SEQ ID NO: 24 |
| | Co+/Co(a−b−) (SNP2) | rs777730687 | SEQ ID NO: 25 | SEQ ID NO: 26 | SEQ ID NO: 27 |
| | Co^{a}/Co^{b} | rs28362692 | SEQ ID NO: 28 | SEQ ID NO: 29 | SEQ ID NO: 30 |
| Cromer | Crom+/Cromerₙᵤₗₗ | rs1131690771 | SEQ ID NO: 31 | SEQ ID NO: 32 | SEQ ID NO: 33 |
| | (SNP1) | | | | |
| (CROM) | Crom+/Cromerₙᵤₗₗ | rs121909603 | SEQ ID NO: 34 | SEQ ID NO: 35 | SEQ ID NO: 36 |
| | (SNP2) | | | | |
| | Crom+/Cromerₙᵤₗₗ | rs762195469 | SEQ ID NO: 37 | SEQ ID NO: 38 | SEQ ID NO: 39 |
| | (SNP3) | | | | |
| | Cr(a+)/Cr(a−) | rs60822373 | SEQ ID NO: 40 | SEQ ID NO: 41 | SEQ ID NO: 42 |
| Diego (DI) | Di^{a}/Di^{b} | rs2285644 | SEQ ID NO: 43 | SEQ ID NO: 44 | SEQ ID NO: 45 |
| Duffy (FY) | Fy^{a}/Fy^{b} | rs12075 | SEQ ID NO: 46 | SEQ ID NO: 47 | SEQ ID NO: 48 |
| Gerbich (GE) | GE+/Leach | GYPC_c_134delC | SEQ ID NO: 49 | SEQ ID NO: 50 | SEQ ID NO: 51 |
| | GE+/GEIS+ | GYPC_c_95C_A | SEQ ID NO: 52 | SEQ ID NO: 53 | SEQ ID NO: 54 |
| I (I) | I+/I− (SNP1) | rs1177742207 | SEQ ID NO: 55 | SEQ ID NO: 56 | SEQ ID NO: 57 |
| | I+/I− (SNP2) | rs56141211 | SEQ ID NO: 58 | SEQ ID NO: 59 | SEQ ID NO: 60 |
| | I+/I− (SNP3) | rs755228157 | SEQ ID NO: 61 | SEQ ID NO: 62 | SEQ ID NO: 63 |
| | I+/I− (SNP4) | rs774740944 | SEQ ID NO: 64 | SEQ ID NO: 65 | SEQ ID NO: 66 |
| | I+/I− (SNP5) | rs201291494 | SEQ ID NO: 67 | SEQ ID NO: 68 | SEQ ID NO: 69 |
| | I+/I− (SNP6) | rs55940927 | SEQ ID NO: 70 | SEQ ID NO: 71 | SEQ ID NO: 72 |
| Indian (IN) | In^{a}/In^{b} | rs369473842 | SEQ ID NO: 73 | SEQ ID NO: 74 | SEQ ID NO: 75 |
| Kidd (JK) | Jk+/Jk(a−b−) (SNP1) | rs538368217 | SEQ ID NO: 76 | SEQ ID NO: 77 | SEQ ID NO: 78 |
| | Jk+/Jk(a−b−) (SNP2) | rs78937798 | SEQ ID NO: 79 | SEQ ID NO: 80 | SEQ ID NO: 81 |
| | Jk^{a}/Jk^{b} | rs1058396 | SEQ ID NO: 82 | SEQ ID NO: 83 | SEQ ID NO: 84 |
| JR (JR) | Jr(a+)/Jr(a−) (SNP1) | rs140207606 | SEQ ID NO: 85 | SEQ ID NO: 86 | SEQ ID NO: 87 |
| | Jr(a+)/Jr(a−) (SNP2) | rs548254708 | SEQ ID NO: 88 | SEQ ID NO: 89 | SEQ ID NO: 90 |
| | Jr(a+)/Jr(a−) (SNP3) | rs72552713 | SEQ ID NO: 91 | SEQ ID NO: 92 | SEQ ID NO: 93 |
| Kell (KEL) | K/k | rs8176058 | SEQ ID NO: 94 | SEQ ID NO: 95 | SEQ ID NO: 96 |
| Knops (KN) | Kn^{a}/Kn^{b} | rs41274768 | SEQ ID NO: 97 | SEQ ID NO: 98 | SEQ ID NO: 99 |
| LAN (LAN) | Lan+/Lan− (SNP1) | rs769584110 | SEQ ID NO: 100 | SEQ ID NO: | SEQ ID NO: |
| | | | | 101 | 102 |
| | Lan+/Lan− (SNP2) | rs202232534 | SEQ ID NO: 103 | SEQ ID NO: | SEQ ID NO: |
| | | | | 104 | 105 |
| | Lan+/Lan− (SNP3) | rs755723161 | SEQ ID NO: 106 | SEQ ID NO: | SEQ ID NO: |
| | | | | 107 | 108 |
| Lutheran (LU) | Lu+/Lu(a−b−) (SNP1) | KLF1_19-12996560- | SEQ ID NO: 109 | SEQ ID NO: | SEQ ID NO: |
| | | T-TG | | 110 | 111 |
| | Lu+/Lu(a−b−) | rs483352838 | SEQ ID NO: 112 | SEQ ID NO: | SEQ ID NO: |
| | (SNP2-P1) | | | 113 | 114 |
| | Lu+/Lu(a−b−) | rs483352838 | SEQ ID NO: 115 | SEQ ID NO: | SEQ ID NO: |
| | (SNP2-P2) | | | 116 | 117 |
| | Lu^{a}/Lu^{b} | rs28399653 | SEQ ID NO: 118 | SEQ ID NO: | SEQ ID NO: |
| | | | | 119 | 120 |
| P1PK (P1PK) | P^{k}+/p (SNP1) | rs1398859071 | SEQ ID NO: 121 | SEQ ID NO: | SEQ ID NO: |
| | | | | 122 | 123 |
| | P^{k}+/p (SNP2) | rs387906280 | SEQ ID NO: 124 | SEQ ID NO: | SEQ ID NO: |
| | | | | 125 | 126 |
| | P^{k}+/p (SNP3) | A4GALT_c_418 | SEQ ID NO: 127 | SEQ ID NO: | SEQ ID NO: |
| | | | | 128 | 129 |
| | P^{k}+/p (SNP4) | A4GALT_ | SEQ ID NO: 130 | SEQ ID NO: | SEQ ID NO: |
| | | MG812384 | | 131 | 132 |
| | P^{k}+/p (SNP5) | rs1189809232 | SEQ ID NO: 133 | SEQ ID NO: | SEQ ID NO: |
| | | | | 134 | 135 |
| | P^{k}+/p (SNP6) | rs755279796 | SEQ ID NO: 136 | SEQ ID NO: | SEQ ID NO: |
| | | | | 137 | 138 |
| | P^{k}+/p (SNP7) | rs778387354 | SEQ ID NO: 139 | SEQ ID NO: | SEQ ID NO: |
| | | | | 140 | 141 |
| H(H) | H+/Para-Bombay | rs777455020 | SEQ ID NO: 142 | SEQ ID NO: | SEQ ID NO: |
| | (SNP1) | | | 143 | 144 |
| | H+/Para-Bombay | rs573412368 | SEQ ID NO: 145 | SEQ ID NO: | SEQ ID NO: |
| | (SNP2) | | | 146 | 147 |
| | H+/Para-Bombay | rs574691621 | SEQ ID NO: 148 | SEQ ID NO: | SEQ ID NO: |
| | (SNP3) | | | 149 | 150 |
| MNS (MNS) | S/s | rs7683365 | SEQ ID NO: 151 | SEQ ID NO: | SEQ ID NO: |
| | | | | 152 | 153 |
| Vel (VEL) | Vel+/Vel− (SNP1) | rs1169340827 | SEQ ID NO: 154 | SEQ ID NO: | SEQ ID NO: |
| | | | | 155 | 156 |
| | Vel+/Vel− (SNP2) | rs554492306 | SEQ ID NO: 157 | SEQ ID NO: | SEQ ID NO: |
| | | | | 158 | 159 |
| | Vel+/Vel− (SNP3) | rs566629828 | SEQ ID NO: 160 | SEQ ID NO: | SEQ ID NO: |
| | | | | 161 | 162 |
| | Vel+/Vel− (SNP4) | rs899095555 | SEQ ID NO: 163 | SEQ ID NO: | SEQ ID NO: |
| | | | | 164 | 165 |
| | Vel+/Vel− (SNP5) | rs1182690110 | SEQ ID NO: 166 | SEQ ID NO: | SEQ ID NO: |
| | | | | 167 | 168 |
| | Vel+/Vel− (SNP6) | rs1207554936 | SEQ ID NO: 169 | SEQ ID NO: | SEQ ID NO: |
| | | | | 170 | 171 |
| Yt(YT) | Yt+/Yt(a−b−) (SNP1) | rs772244054 | SEQ ID NO: 172 | SEQ ID NO: | SEQ ID NO: |
| | | | | 173 | 174 |
| | Yt+/Yt(a−b−) (SNP2) | rs114782198 | SEQ ID NO: 175 | SEQ ID NO: | SEQ ID NO: |
| | | | | 176 | 177 |
| | Yt+/Yt(a−b−) (SNP3) | rs771039143 | SEQ ID NO: 178 | SEQ ID NO: | SEQ ID NO: |
| | | | | 179 | 180 |
| | Yt+/Yt(a−b−) (SNP4) | ACHE | SEQ ID NO: 181 | SEQ ID NO: | SEQ ID NO: |
| | | | | 182 | 183 |
| | Yt^{a}/Yt^{b} | rs1799805 | SEQ ID NO: 184 | SEQ ID NO: | SEQ ID NO: |
| | | | | 185 | 186 |

Notes:
1) The names of the blood group systems are named according to the International Society of Blood Transfusion (ISBT), and system names (system symbols) are listed in Table of blood group systems v. 10.0.
2) In the phenotype, “blood group system symbol +”, for example, Co+, I+, etc., indicates that a protein corresponding to a gene encoded by the blood group system is in a wild type.
3) For the expression of other phenotypes and blood group antigens in the phenotype, refer to The Blood Group Antigen FactsBook (Third Edition).

3. Detection Steps

1) PCR Amplification

By using all amplification primer combinations (including forward primers and reverse primers) shown in Table 8, samples to be detected obtained in Step 1 are amplified by multiplex PCR to obtain target sequence amplification products of the samples to be detected. Reagents in Table 9 and All primers in Table 8 are placed in one amplification tube (the amplification tube may be replaced with 96-well plates, each well corresponds to one sample, the reagents in each well are the same, and the conditions are the same) for amplification, and each amplification tube corresponds to one sample. In this embodiment, there are 155 amplification tubes for simultaneous amplification.

A PCR amplification reaction system is shown in Table 9.

TABLE 9
Multiplex PCR amplification reaction system

| Components | Volume (μL) |
|---|---|
| Water, HPLC grade | 0.8 |
| 10 × PCR Buffer with 20 mM MgCl₂ | 0.5 |
| 25 mM MgCl₂ | 0.4 |
| 25 mMdNTP Mix (dNTP mix) | 0.1 |
| 0.2 to 2.0 uM Primer Mix (primer | 1 |
| combination, Table 8) | |
| 5 U/μl PCR Enzyme (PCR polymerase) | 0.2 |
| 5 to 20 ng/μL DNA (DNA to be detected) | 2 |
| Total volume | 5 |

Cycle conditions of PCR amplification reaction are as follows: 95° C., 2 minutes; 45 cycles: 95° C., 30 seconds, 56° C., 30 seconds, 72° C., 60 seconds, 72° C., 5 minutes; keeping a temperature of 4° C.

2) Treatment with a Shrimp Alkaline Phosphatase (SAP)

After amplification, remaining dNTPs are treated by the shrimp alkaline phosphatase (SAP) to prevent interference with subsequent base extension. An SAP premixed solution system is shown in Table 10.

TABLE 10
SAP premixed solution system

| Components | Volume (μL) |
|---|---|
| Nanopure Water, Autoclaved (ultrapure | 1.53 |
| water) | |
| SAP Buffer | 0.17 |
| SAP Enzyme (1.7 U/ul) (shrimp alkaline | 0.30 |
| phosphatase) | |
| Total volume | 2 |

In Step 1), 2 μl of an SAP premixed solution is added to each amplification tube after PCR amplification, a total volume after the mixed solution is added is 7 and then SAP reaction is conducted in an amplification instrument. Reaction programs are as follows: 37° C., 40 minutes; 85° C., 5 minutes; keeping a temperature of 4° C.

3) Base Extension

By using all extension primer combinations shown in Table 8, purified products in Step 2) are amplified by single-base extension. Through this amplification, a sequence-specific single base is extended at a 3′ end of an extension probe as a molecular weight marker. A single base extension premixed solution system is shown in Table 11.

TABLE 11
Extension premixed solution system

| Components | Volume (μL) |
|---|---|
| Nanopure Water, Autoclaved (ultrapure | 0.619 |
| water) | 0.200 |
| iPLEX Buffer (extension buffer) | |
| iPLEX Termination Mix (extension | 0.200 |
| termination mix) | 0.94 |
| Extend Primer Mix (extension primer | |
| combination) | 0.041 |
| iPLEX Enzyme (single-base extension | |
| reaction enzyme) | |
| Total volume | 2 |

In Step 2), 2 μl of an extension premixed solution is added to each amplification tube after treatment with the shrimp alkaline phosphatase (SAP), a total volume after the mixed solution is added is 9 and then extension reaction is conducted in an amplification instrument.

Single-base extension reaction programs are as follows: 95° C., 30 seconds; (95° C., 5 seconds; (52° C., 5 seconds, 80° C., 5 seconds; 5 cycles) 40 cycles); 72° C., 3 minutes; keeping a temperature of 4° C.

4) Desalination with Resin

41 μl of HPLC water is added to each amplification tube, resin is used for sample desalination, and extension reaction products are purified.

5) Mass spectrometry detection Samples are subjected to sample application onto a chip (Manufacturer: Agena Bioscience, Model: SpectroCHiP CPM96). Molecular weight detection is performed by a mass spectrometer to determine the species of specific bases and the type of samples to be detected.

6) Result Analysis

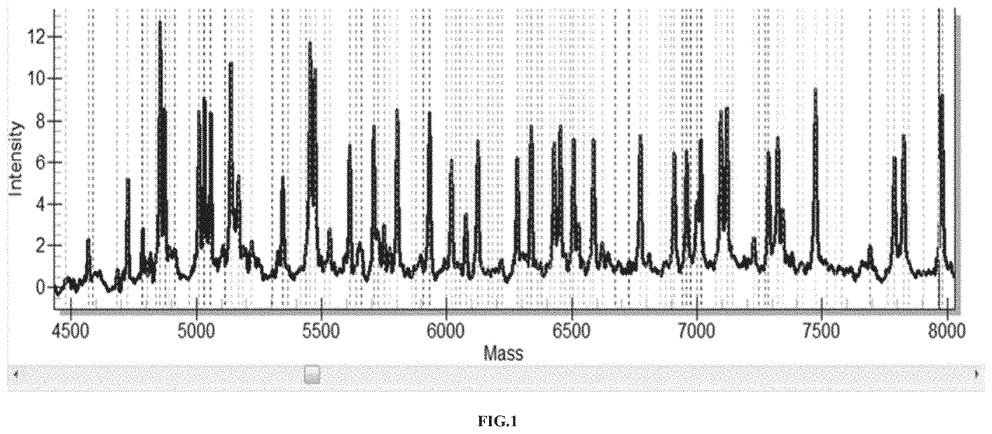

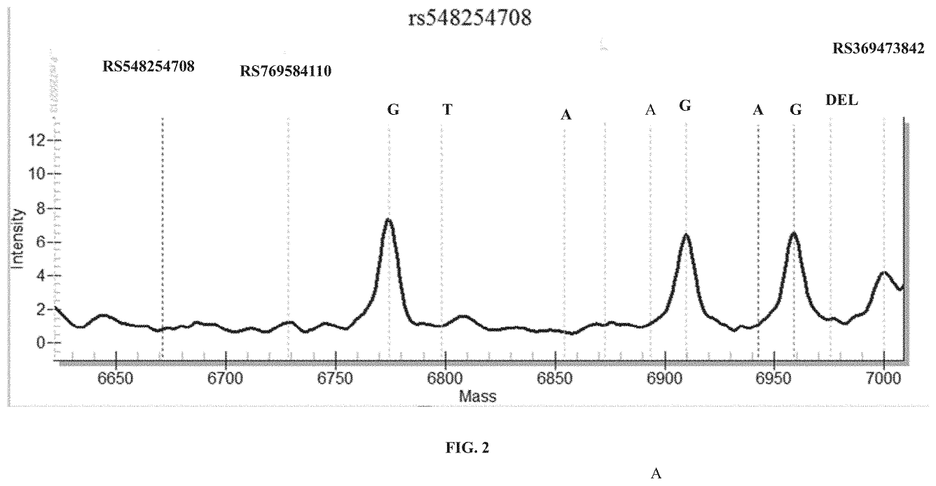

Mass spectrometry detection is performed on the 155 cases of samples, and all sites have good results in all the samples (mass spectrometry software is rated A (Conservative) or B (Mordarate)). An obtained representative detection mass spectrogram is shown in FIG. 1. Among them, a detection mass spectrogram of an rs548254708 site is shown in FIG. 2. For 36 cases of randomly selected samples, serological recheck is performed on antigen gene sites for which corresponding blood group antibodies can be obtained. Blood group antibodies used include: anti-C, -c, -E, -e, -S, -s, -K, -k, -Fy^{a}, -Fy^{b}, -jk^{a}, -jk^{b}, -Lu^{a}, -Lu^{b} and -CD59. Sequencing recheck is performed on antigen gene sites without blood group antibodies. Results of mass spectrometry-based erythrocyte blood groups are completely consistent with serological or sequencing results. Sensitivity=true positive results/(true positive results+false negative results)*100%=100%. Specificity=the number of true negatives/(the number of true negatives+the number of false positives)*100%=100%.

TABLE 12
Statistics of serological verification results

| Blood | | | | | | | | |
|---|---|---|---|---|---|---|---|---|
| group | Pheno | SNP | | | | | | |
| systems | type | sites | Serological results | | | Mass spectrometry results | | |
| Rh | C/c | rs586178 | C+c- | C-c+ | C+c+ | GG→C+c- | CC→C-c | GC→C+c+ |
| (RH) | | | →15 | →6 | →15 | →15 | +→6 | →15 |
| | E/e | rs609320 | E+e- | E-e+ | E+e+ | GG→E+e- | CC→E-e+ | GC→E+e+ |
| | | | →3 | →17 | →16 | →17 | →3 | →16 |
| CD59 | CD59: | CD59_c_ | CD59: | CD59 | / | ins/ins→C | del/del→ | ins/del→C |
| (CD59) | +1/ | 361delG | +1→3 | :-11→ | | D59:+1→36 | CD59:-1 | D59:+1→ |
| | CD59: | | 6 | 0 | | | →0) | 0 |
| | -1 | CD59_c_ | | | | ins/ins→C | del/del→ | ins/del→C |
| | | 123delC | | | | D59:+1→36 | CD59:-1 | D59:+1→ |
| | | | | | | | →0 | 0 |
| Duffy | Fyª/Fy^{b} | rs12075 | Fy(a+ | Fy(a- | Fy(a+ | GG→Fy(a | AA→Fy(a | GA→Fy(a |
| (FY) | | | b-)→ | b+)→ | b+)→ | +b-)→33 | -b+)→0 | +b+)→3 |
| | | | 33 | 0 | 3 | | | |
| Kidd | Jk+/Jk | rs538368217 | Jk+→ | Jk(a-b-) | / | GG→Jk+ | AA→Jk(a | GA→Jk+ |
| (JK) | (a-b-) | | 36 | →0 | | →36 | -b-)→0 | →0 |
| | | rs78937798 | Jk+> | Jk(a-b-) | / | GG→Jk+ | AA→Jk(a | GA→Jk+ |
| | | | 36 | →0 | | →36 | -b-)→0 | →0 |
| | Jkª/Jk^{b} | rs1058396 | Jk(a+b | Jk(a-b+) | Jk(a+b+) | GG→Jk(a+b-) | AA→Jk(a | GA→Jk(a+b+) |
| | | | -)→8 | →11 | →17 | →8 | -b+)→11 | →17 |
| Kell | K/k | rs8176058 | K-k+ | K+k- | K+k+ | GG→K-k+ | AA→K+k- | GA→K+k |
| (KEL) | | | →36 | →0 | →0 | →36 | →0 | +→36 |
| Lutheran | Lu+/ | KLF1_19-12 | Lu+→ | Lu(a- | / | del/del→ | ins/ins→ | del/ins→L |
| (LU) | Lu(a-b-) | 996560-T- | 36 | b-)→ | | Lu+→36 | ND→0 | u(a-b-)→0 |
| | | TG | | 0 | | | | |
| | | rs483352838 | Lu+→ | Lu(a- | / | del/del→ | ins/ins→ | del/ins→L |
| | | | 36 | b-)→ | | Lu+→36 | ND→0 | u(a-b-)→0 |
| | | | | 0 | | | | |
| | Luª/Lu^{b} | rs28399653 | Lu(a-b | Lu(a+ | Lu(a+ | GG→Lu(a | AA→Lu | GA→Lu |
| | | | +)→36 | b-)→ | b+)→ | -b+)→36 | (a+b-)→0 | (a+b+)→0 |
| | | | | 0 | 0 | | | |
| MNS | S/s | rs7683365 | S+s- | S-s+ | S+s+ | AA→S+s- | GG→S-s+ | AG→S+s |
| (MAS) | | | →0 | →36 | →0 | →0 | →36 | +→0 |

TABLE 13
Statistics of sequencing verification results

| Blood | | | | | | | | |
|---|---|---|---|---|---|---|---|---|
| group | Pheno- | SNP | | | | | | |
| systems | type | sites | Sequencing results | | | Mass spectrometry results | | |
| Augustine | At(a+)/ | rs775471940 | ins/ins→ | del/del | ins/del→ | ins/ins→ | del/del | ins/del→ |
| (AUG) | At(a-) | | 36 | →0 | 0 | 36 | →0 | 0 |
| | | rs45458701 | GG→36 | AA→0 | GA→0 | GG→36 | AA→0 | GA→0 |
| | | rs759118384 | ins/ins→ | del/del | ins/del→ | ins/ins→ | del/del | ins/del→ |
| | | | 36 | →0 | 0 | 36 | →0 | 0 |
| Colton | Co+/ | rs749625062 | ins/ins→ | del/del | ins/del→ | ins/ins→ | del/del | ins/del→ |
| (CO) | Co(a-b-) | | 36 | →0 | 0 | 36 | →0 | 0 |
| | | rs777730687 | ins/ins→ | del/del | ins/del→ | ins/ins→ | del/del | ins/del→ |
| | | | 36 | →0 | 0 | 36 | →0 | 0 |
| | Co^{a}/Co^{b} | rs28362692 | CC→36 | TT→0 | CT→0 | CC→36 | TT→0 | CT→0 |
| Cromer | Crom+/ | rs1131690771 | CC→36 | AA→0 | CA→0 | CC→36 | AA→0 | CA→0 |
| (CROM) | Cromerₙᵤₗₗ | rs121909603 | GG→36 | AA→0 | GA→0 | GG→36 | AA→0 | GA→0 |
| | | rs762195469 | CC→36 | TT→0 | CT→0 | CC→36 | TT→0 | CT→0 |
| | Cr(a+)/ | rs60822373 | GG→36 | CC→0 | GC→36 | GG→36 | CC→0 | GC→36 |
| | Cr(a-) | | | | | | | |
| Diego | Diª/Di^{b} | rs2285644 | GG→36 | AA→0 | GA→0 | GG→36 | AA→0 | GA→0 |
| (DI) | | | | | | | | |
| Gerbich | GE+/ | GYPC_ | ins/ins→ | del/del | ins/del→ | ins/ins→ | del/del | ins/del→ |
| (GE) | Leach | c_134delC | 36 | →0 | 0 | 36 | →0 | 0 |
| | GE+/ | GYPC | CC→36 | AA→0 | CA→0 | CC→36 | AA→0 | CA-0 |
| | GEIS | c_95C_ | | | | | | |
| | + | A | | | | | | |
| I | I+/I- | rs1177742207 | GG→36 | AA→0 | GA→0 | GG→36 | AA→0 | GA→0 |
| (I) | | rs56141211 | GG→36 | AA→0 | GA→0 | GG→36 | AA→0 | GA→0 |
| | | rs755228157 | GG→36 | AA→0 | GA→0 | GG→36 | AA→0 | GA→0 |
| | | rs774740944 | GG→36 | AA→0 | GA→0 | GG→36 | AA→0 | GA→0 |
| | | rs201291494 | TT→36 | AA→0 | TA→0 | TT→36 | AA→0 | TA→0 |
| | | rs55940927 | GG→36 | AA→0 | GA→0 | GG→36 | AA→0 | GA→0 |
| Indian | Inª/In^{b} | rs369473842 | GG→36 | CC→0 | GC→0 | GG→36 | CC→0 | GC→0 |
| (IN) | | | | | | | | |
| JR | Jr(a+)/ | rs140207606 | GG→36 | AA→0 | GA→0 | GG→36 | AA→0 | GA→0 |
| (JR) | Jr(a-) | rs548254708 | GG→36 | AA→0 | GA→0 | GG→36 | AA→0 | GA→0 |
| | | rs72552713 | GG→36 | AA→0 | GA→0 | GG→36 | AA→0 | GA→0 |
| Knops | Knª/Kn^{b} | rs41274768 | GG→36 | AA→0 | GA→0 | GG→36 | AA→0 | GA→0 |
| (KN) | | | | | | | | |
| LAN | Lan+/ | rs769584110 | ins/ins→ | del/del | ins/del→ | ins/ins→ | del/del | ins/del→ |
| (LAN) | Lan- | | 36 | →0 | 0 | 36 | →0 | 0 |
| | | rs202232534 | GG→36 | TT→0 | GT→0 | GG→36 | TT→0 | GT→0 |
| | | rs755723161 | ins/ins→ | del/del | ins/del→ | ins/ins→ | del/del | ins/del→ |
| | | | 36 | →0 | 0 | 36 | →0 | 0 |
| P1PK | Pk+/p | rs1398859071 | ins/ins→ | del/del | ins/del→ | ins/ins→ | del/del | ins/del→ |
| (P1PK) | | | P^{k}+36 | →p→0 | P^{k}+→0 | P^{k}+→36 | →p→0 | P^{k}+→0 |
| | | rs387906280 | del/del→ | ins/ins→ | del/ins→ | del/del→ | ins/ins→ | del/ins→ |
| | | | P^{k}+→36 | p→0 | P^{k}+→0 | P^{k}+→36 | p→0 | P^{k}+→0 |
| | | A4GALT_ | InsShort/ | InsLong/ | InsShort/ | InsShort/ | InsLong/ | InsShort/ |
| | | c_418 | InsShort→ | InsLong | InsLong→ | InsShort→ | InsLong | InsLong→ |
| | | | P^{k}+→36 | →p→0 | P^{k}+→0 | P^{k}+→36 | →p→0 | P^{k}+→0 |
| | | A4GALT_ | del/del→ | ins/ins→ | del/ins→ | del/del→ | ins/ins→ | del/ins→ |
| | | MG812384 | P^{k}+→36 | p→0 | P^{k}+→0 | P^{k}+→36 | →p→0 | P^{k}+→0 |
| | | rs1189809232 | ins/ins→ | del/del | ins/del→ | ins/ins→ | del/del | ins/del→ |
| | | | P^{k}+36 | →p→0 | P^{k}+→0 | P^{k}+→36 | →p→0 | P^{K}+→0 |
| | | rs755279796 | TT→P^{k}+ | AA→p | TA→P^{k}+ | TT→P^{k}+ | AA→p | TA→P^{k}+ |
| | | | →36 | →0 | →0 | →36 | →0 | →0 |
| | | rs778387354 | ins/ins→ | del/del | ins/del→ | ins/ins→ | del/del | ins/del→ |
| | | | P^{k}+36 | →p→0 | P^{k}+→0 | P^{k}+36 | →p→0 | P^{k}+→0 |
| H | H+/Para- | rs777455020 | ins/ins→ | del/del | ins/del→ | ins/ins→ | del/del | ins/del→ |
| (H) | Bombay | | 36 | →0 | 0 | 36 | →0 | 0 |
| | | rs573412368 | ins/ins→ | del/del | ins/del→ | ins/ins→ | del/del | ins/del→ |
| | | | 36 | →0 | 0 | 36 | →0 | 0 |
| | | rs574691621 | GG→36 | AA→0 | GA→0 | GG→36 | AA→0 | GA→0 |
| Vel | Vel+/ | rs1169340827 | GG→36 | AA→0 | GA→0 | GG→36 | AA→0 | GA→0 |
| (EL) | Vel- | rs554492306 | GG→36 | AA→0 | GA→0 | GG→36 | AA→0 | GA→0 |
| | | rs566629828 | ins/ins→ | del/del | ins/del→ | ins/ins→ | del/del | ins/del→ |
| | | | 36 | →0 | 0 | 36 | →0 | 0 |
| | | rs899095555 | CC-+36 | TT→0 | CT→0 | CC→36 | TT→0 | CT→0 |
| | | rs1182690110 | TT→36 | AA,GG, | TA,TG→ | TT→36 | AA,GG, | TA,TG→ |
| | | | | AG→0 | 0 | | AG→0 | 0 |
| | | rs1207554936 | ins/ins→ | del/del | ins/del→ | ins/ins→ | del/del→ | ins/del→ |
| | | | 36 | →36 | 36 | 36 | 36 | 36 |
| Yt | Yt+/Yt | rs772244054 | ins/ins→ | del/del | ins/del→ | ins/ins→ | del/del | ins/del→ |
| (YT) | (a-b-) | | 36 | →0 | 0 | 36 | →0 | 0 |
| | | rs114782198 | GG→36 | AA→0 | GA→0 | GG→36 | AA→0 | GA→0 |
| | | rs771039143 | del/del→ | ins/ins→ | del/ins→ | del/del→ | ins/ins→ | del/ins→ |
| | | | 350 | 0 | 0 | 36 | 0 | 0 |
| | | ACHE | ins/ins→ | del/del | ins/del→ | ins/ins→ | del/del | ins/del→ |
| | | | 36 | →0 | 0 | 36 | →0 | 0 |
| | Yt^{a}/Yt^{b} | rs1799805 | GG→36 | TT→0 | GT→0 | GG→36 | TT→0 | GT→0 |

Embodiment 2 Exploration of detection methods of blood group C site In this embodiment, an initially designed detection target for a blood group C/c is rs676785. During preliminary verification, it is found that rs676785 cannot get detection results very well. The reason may be that there are highly homologous sequences in a DNA region where the rs676785 site is located. After incorporation into an RBC panel (erythrocyte gene combination), while mass spectrometry-based detection is performed on rs676785, the situations are prone to occurring that some sites do not have peaks and are not detected, or it is easy to amplify to homologous sequences of the DNA region where the rs676785 site is located to generate erroneous results, etc., After a lot of experimental screening, rs586178 is finally selected as a detection target.

Initial amplification and extension primers for rs676785:

Upstream:
ACGTTGGATGCGAAACTCCGTCTCAAAAAA
Downstream:
ACGTTGGATGCTTGGGCTTCCTCACCTCAAA
UEP (extension primer):
CTGAGCCAGTTCCCT

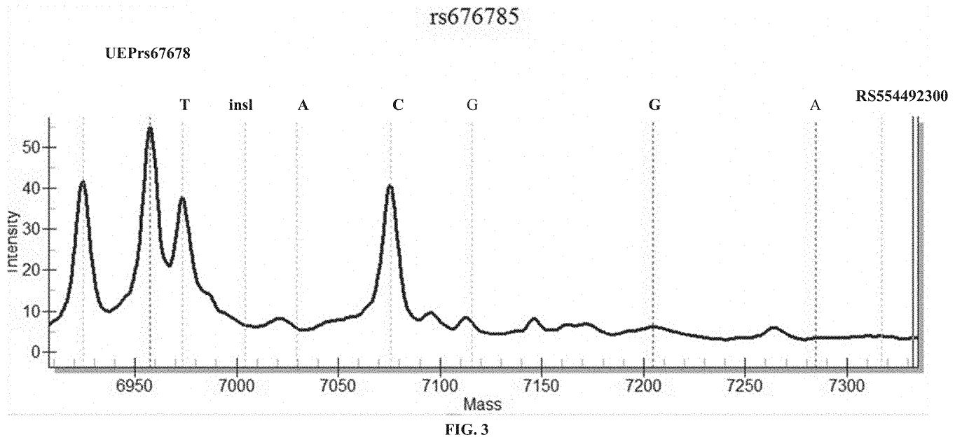

rs676785 is amplified and extended by using the primers. Through mass spectrometry detection, results are shown in FIG. 3.

Amplification and extension primers after replacement with rs586178:

Upstream(forward):
(SEQ ID NO: 10)
ACGTTGGATGAGGCCAGCACAGCCAGCCTTG
Downstream(reverse):
(SEQ ID NO: 11)
ACGTTGGATGATTTGCTCCTGTGACCACTG
UEP(extension):
(SEQ ID NO: 12)
TaTGTCCGGCGCTGCCTGCCCCTCTG

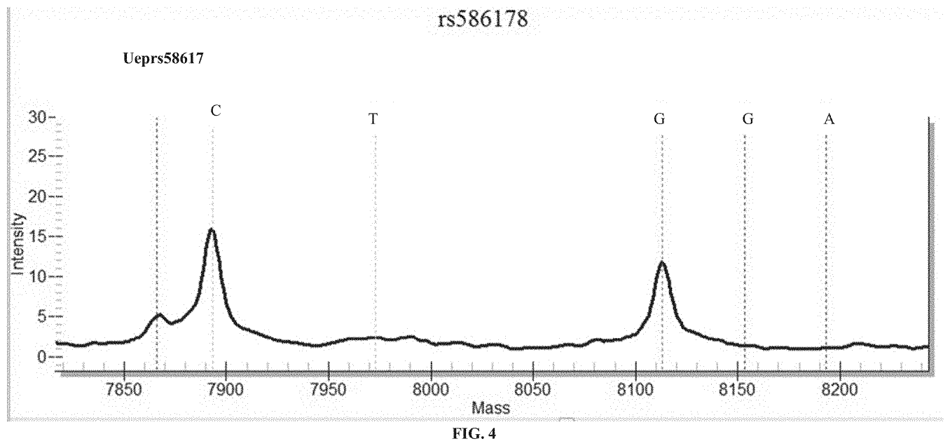

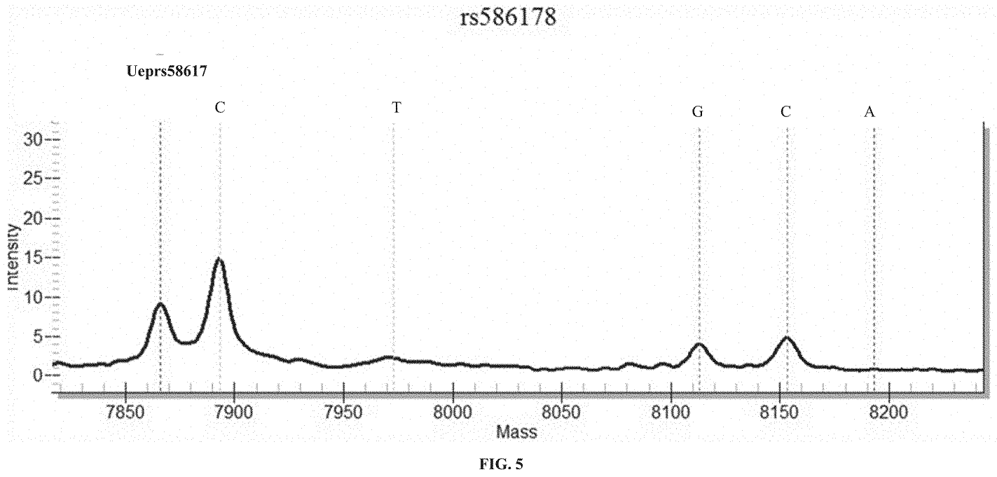

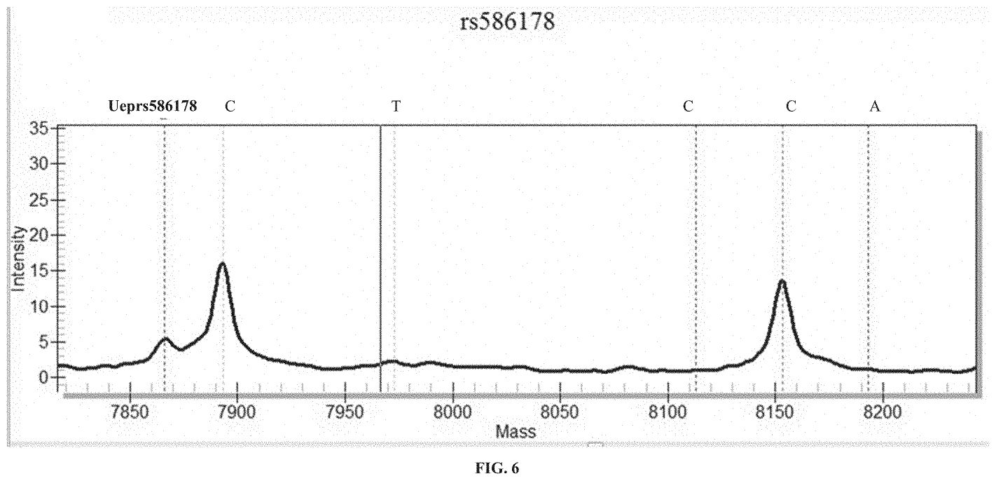

rs586178 is amplified and extended by using the primers. Through mass spectrometry detection, results are shown in FIGS. 4, 5 and 6. FIG. 4 shows homozygous GG→C+c−, with a peak at 8113, FIG. 5 shows heterozygous GG→C+c−, with peaks at 8113 and 8153, and FIG. 6 shows homozygous CC→C−c+, with a peak at 8153.

It can be seen from FIG. 3 that for the blood group C/c, the rs676785 site is used as the detection target, and when it is incorporated into the RBC panel for multiplex (61) PCR, it does not have peaks and is not detected due to the presence of highly homologous sequences, however, after it is replaced with the rs586178 site, stable and correct detection results can be obtained. Before it is replaced with the rs586178 site, the inventor also tries many other sites. During incorporation into the RBC panel for multiplex PCR, they all have the problems such as unstable detection results, no peaks and being undetected, and until it is finally replaced with the rs586178 site, the problem of genotyping detection of the blood group C/c is solved finally.

### Embodiment 3 Selection of Primer Concentrations and Determination of 61 Blood Group Genetic Sites

In this embodiment, an RBC panel is designed on the basis of detection by using the rs586178 site for the blood group C/c determined in Embodiment 2, and 62 sites are initially designed to be detected (in addition to 61 sites listed in the specification, rs75731670 is also included). Results show that after replacement with rs586178, detection of a blood group Lu(a-b-) is affected, making KLF1_19-12996560-T-TG and rs483352838 unstable peak appearance, and meanwhile, detection of an rs75731670 site is also affected. In this embodiment, concentrations of amplification primers of these three sites are adjusted (concentrations of the primers before adjustment are 0.04 to 0.4 μM (final concentration)). It is found that when a concentration of amplification primers of KLF1_19-12996560-T-TG is adjusted to 3 times its original concentration (0.04 to 0.4 μM), and a concentration of rs483352838 is adjusted to 2 times its original concentration (0.04 to 0.4 μM), detection results can be obtained. However, after a concentration of primers of rs75731670 is adjusted many times, stable detection results cannot be obtained. Therefore, the detection of rs75731670 is removed from a system, making the RBC panel become 61 sites.

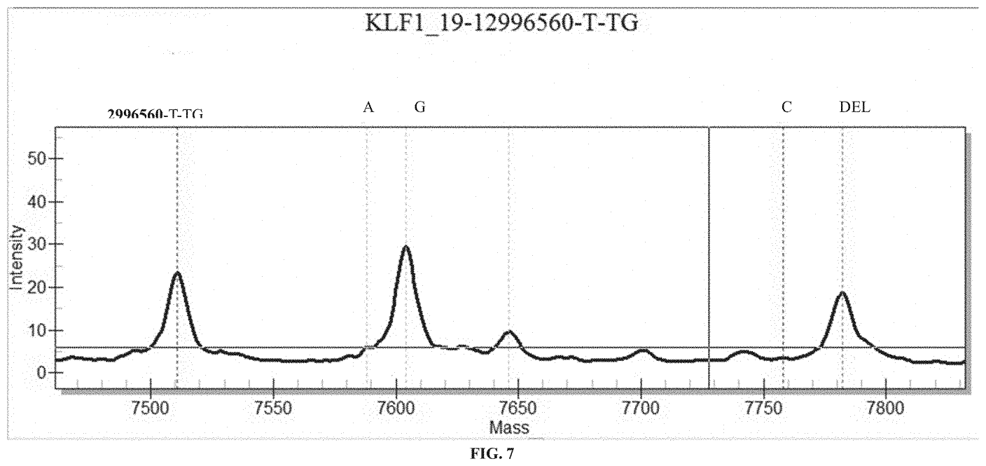

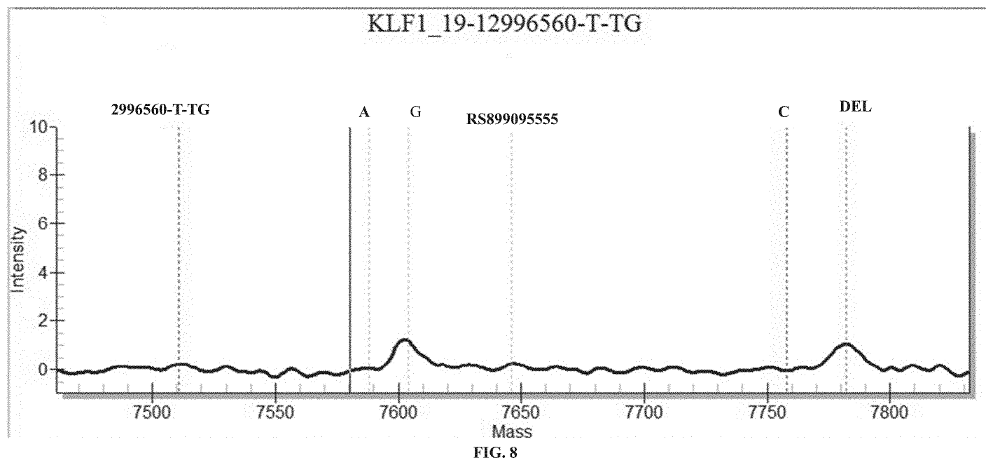

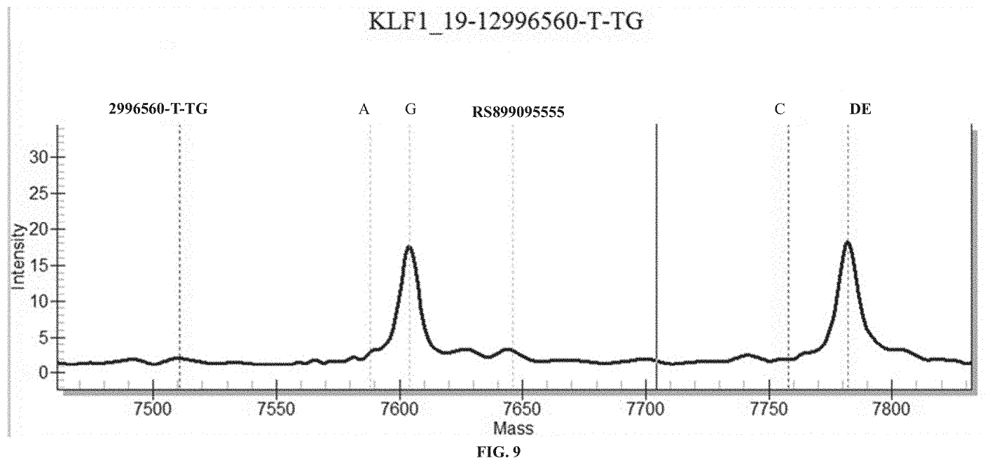

Detection spectrograms of KLF1_19-12996560-T-TG before and after replacement with rs586178 are shown in FIG. 7 and FIG. 8 respectively. It can be seen that before replacement with rs586178, KLF1_19-12996560-T-TG has a higher peak value at 7782, and after replacement with rs586178, the peak value of KLF1_19-12996560-T-TG at 7782 is decreased significantly. A detection spectrogram after a concentration of amplification primers of KLF1_19-12996560-T-TG is adjusted to 3 times the original concentration is shown in FIG. 9. The peak value of KLF1_19-12996560-T-TG at 7782 is significantly increased, which can be used for genotyping detection.

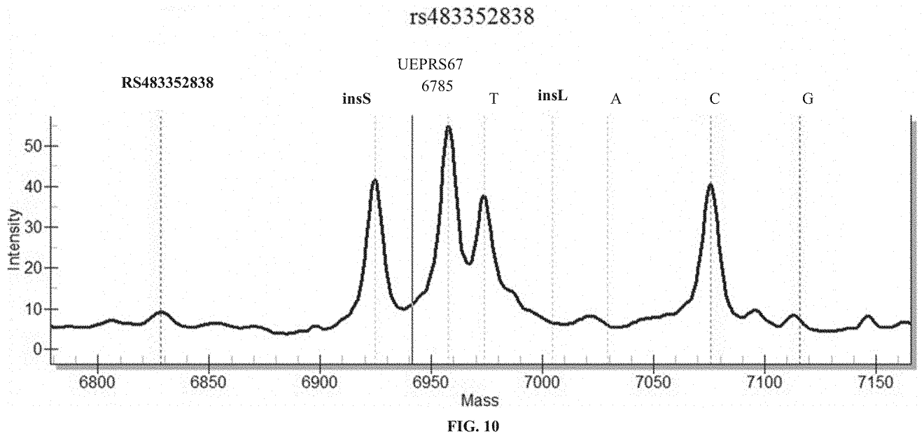

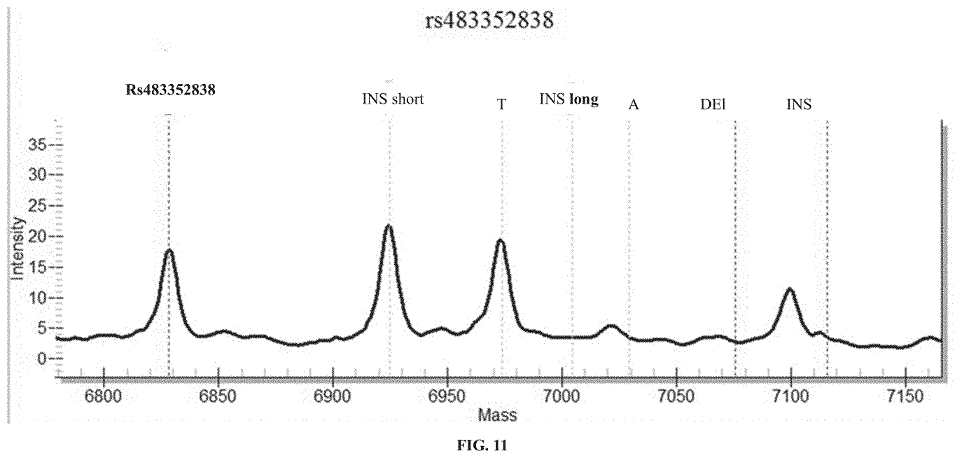

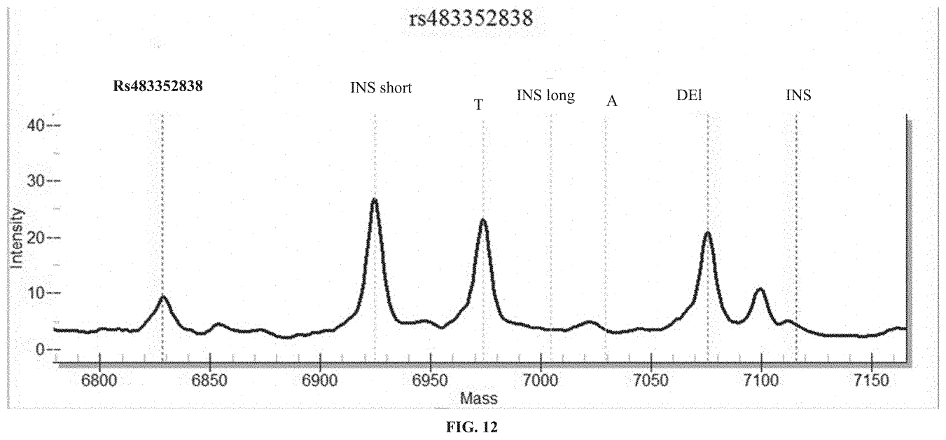

Detection spectrograms of rs483352838 before and after replacement with rs586178 are shown in FIG. 10 and FIG. 11 respectively. It can be seen that before replacement with rs586178, rs483352838 has a higher peak value at 7076, and after replacement with rs586178, the peak value of rs483352838 at 7076 is decreased significantly. A detection spectrogram after a concentration of amplification primers of rs483352838 is adjusted to 2 times the original concentration (0.04 to 0.4 μM) is shown in FIG. 12. The peak value of rs483352838 at 7076 is significantly increased, which can be used for genotyping detection.

### Embodiment 4 Influence of Adjustment of Amplification Primers of Rs7683365 Site of Blood Group S/s on Detection Results

On the basis of Embodiment 3, the RBC panel continues to be optimized. In a follow-up verification process, it is found that in detection results of an rs7683365 site of a blood group S/s, two peak values of heterozygous peaks are quite different, which easily leads to misjudgment. Multiple research experiments are performed on amplification primers, and the amplification primers are then replaced with a group of more appropriate primers:

Initial amplification primers for rs7683365:

Upstream(forward):
ACGTTGGATGGAAACCCGCAGAACAGTTTG
Downstream(reverse):
ACGTTGGATGACAGTGAAACGATGGACAAG

Finally replaced with:

Upstream(forward):
(SEQ ID NO: 151)
ACGTTGGATGTGATTAAGAAAAGGAAACCCG
Downstream(reverse):
(SEQ ID NO: 152)
ACGTTGGATGGGCTTGGCCTCCCAAAATTAT

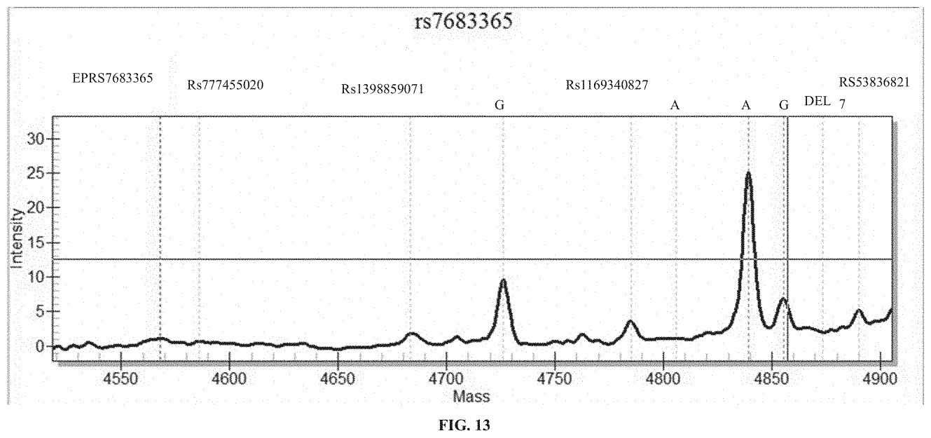

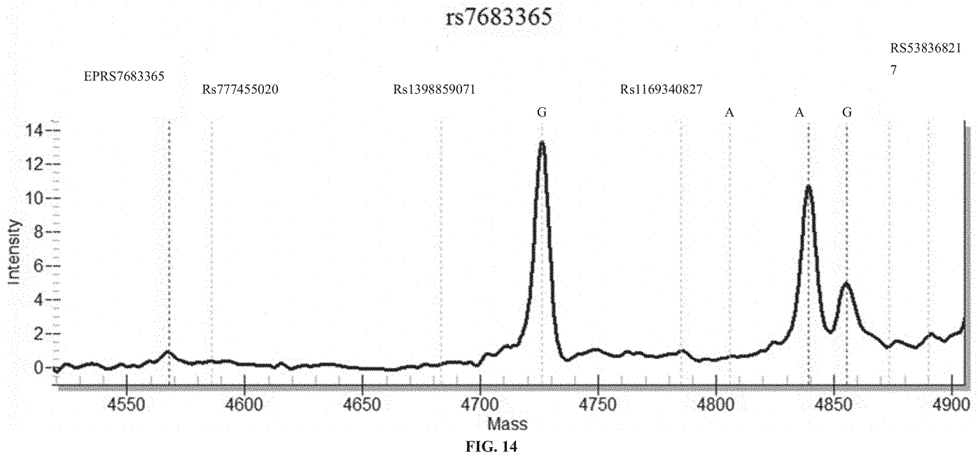

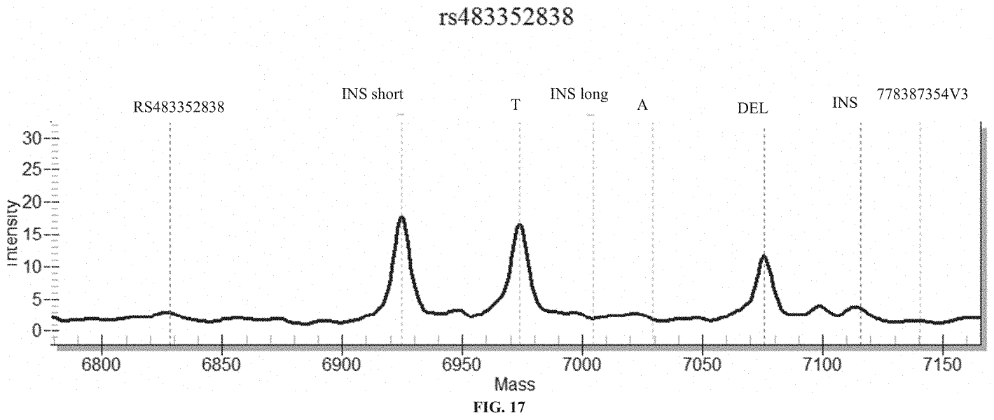

Stable results can be obtained after replacement. Detection spectrograms are shown in FIG. 13 and FIG. 14. FIG. 13 is a detection spectrogram of rs7683365 before amplification primers are replaced, and FIG. 14 is a detection spectrogram of rs7683365 after the amplification primers are replaced. Although FIG. 17 shows that two peaks (at 4839 and 4855) of heterozygotes are still unequal in height, through position distribution of multiple specific samples in a result graph, angles of dividing lines for dividing respective regions are adjusted, so that true values of experiments can be defined, and correct results can be obtained.

### Embodiment 5 Influence of Adjustment of Extension Primers of Rs778387354 Site of Blood Group P^{k}+/p on Detection Results

On the basis of Embodiment 4, accuracy of the system is further verified, the RBC panel continues to be designed, and it is found that detection results of an rs778387354 site of a blood group P^{k}+/p cannot be displayed correctly. From analysis of the detection results, it is found that extension primers of rs778387354 are prone to errors in existing RBC panels used. After multiple primer adjustments, the extension primers of rs778387354 are replaced.

Initial extension primers for rs778387354:

UEP(extension): cccagtCCACGTCCAGGGCAC

Finally replaced with:

(SEQ ID NO: 141)
UEP(extension): TGGAACAAGAAGAGCCAGGGCAC

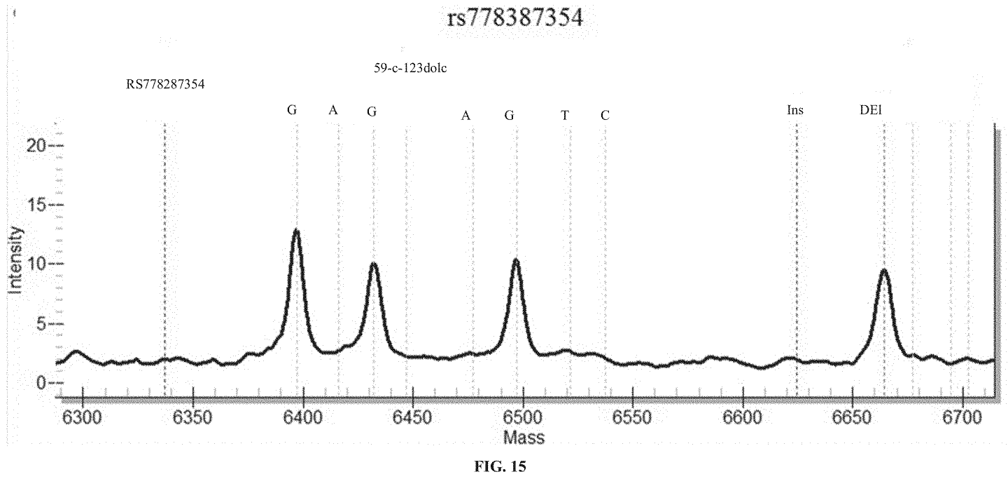

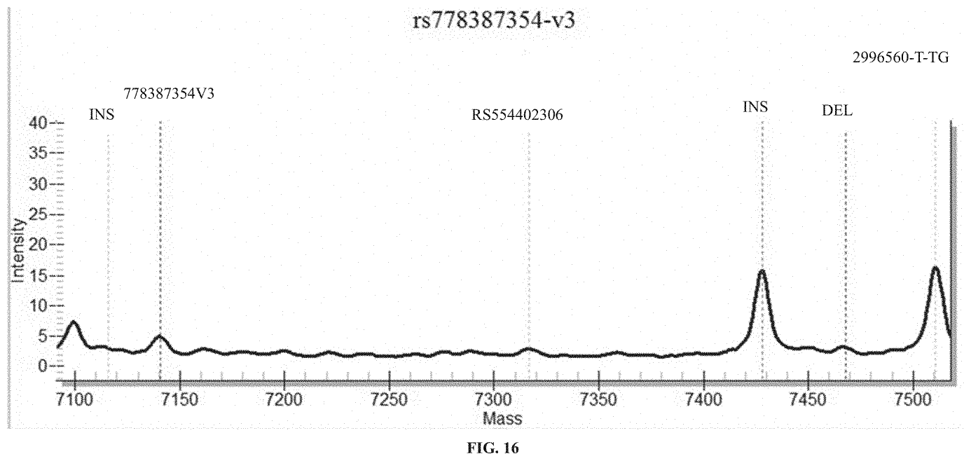

Through verification, correct results can be obtained. Detection results are shown in FIG. 15 and FIG. 16. FIG. 15 is a detection spectrogram before the extension primers are replaced, and there is no peak at an expected peak appearance location 6624 (a non-specific peak appeared at 6664). FIG. 16 is a detection spectrogram after the extension primers are replaced, and there is a peak at 7428, which can be used for detection of genotyping of the rs778387354 site of the blood group P^{k}+/p.

Embodiment 6 Detection methods and primer selection of rs483352838 site On the basis of Embodiment 5, the accuracy of the system is further verified, and the RBC panel continues to be optimized After an rs483352838 site of a blood group Lu(a-b-) is analyzed, it is found that this detection site is a region rich in GC and containing repetitive sequences, as a result, UEP extension primers targeting this site will bind to sequences other than SNP sites to be detected. Subsequently, multiple UEPs are tried to detect this site, and it is found that all primers cannot show stable results.

UEP (Extension Primer) sequences of rs483352838:

(SEQ ID NO: 117)
rs483352838-v1: gCCCGAGGAGgCGGCGCCGGGC
rs483352838-v2: aGAGCCGGCGCCGGGCGCCGGG
rs483352838-v3: aaGTGTACCCGGGGCCCGGCGCC
(SEQ ID NO: 114)
rs483352838-v4: ttattGAGCCGGCGCCGGGCGCCGGG

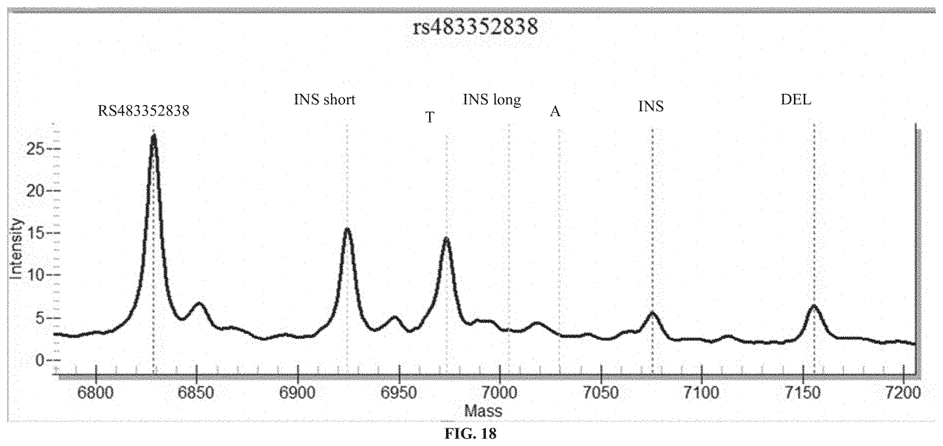

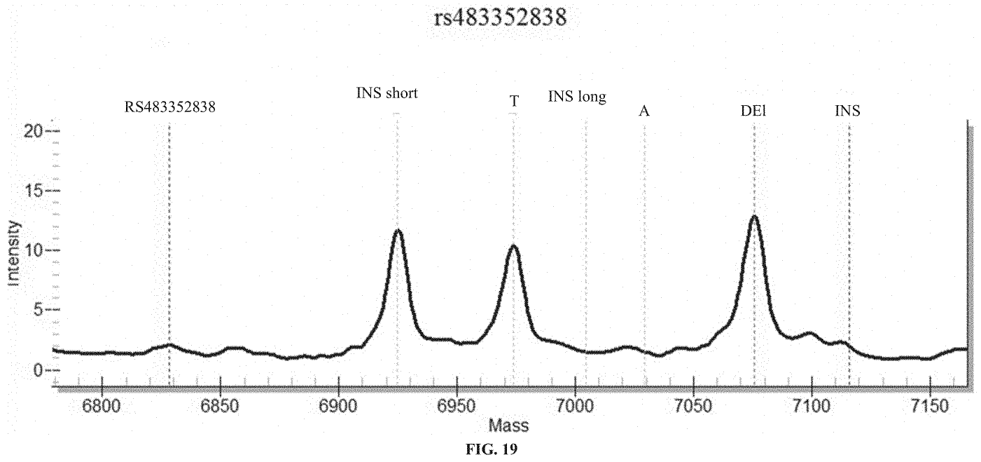

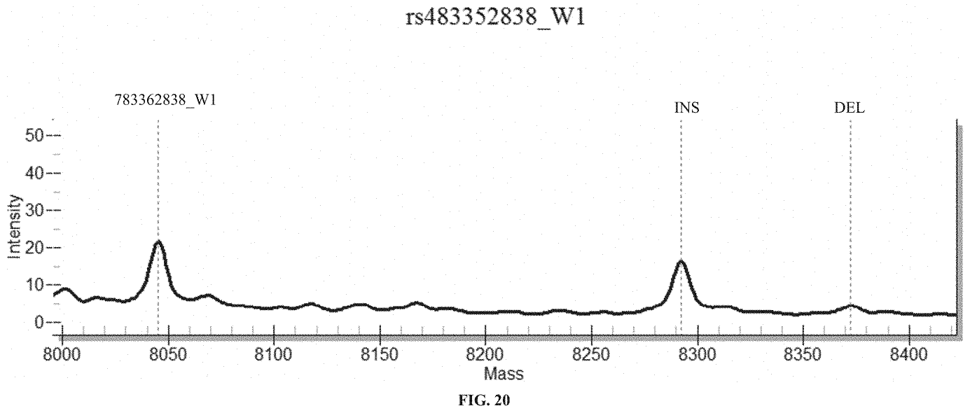

When four extension primers are respectively used for single-base extension, obtained detection spectrograms are shown in FIGS. 17 to 20. FIG. 17 is a detection mass spectrogram obtained during single-base extension of an extension primer rs483352838-v1; FIG. 18 is a detection mass spectrogram obtained during single-base extension of an extension primer rs483352838-v2; FIG. 19 is a detection mass spectrogram obtained during single-base extension of an extension primer rs483352838-v3; and FIG. 20 is a detection mass spectrogram obtained during single-base extension of an extension primer rs483352838-v4.

It can be seen from FIGS. 17 to 20 that rs483352838-v1 can correctly display results of all wild-type samples (there is a peak at 7076), meanwhile, rs483352838-v4 can correctly display results of all mutant samples (there is a peak at 8292), however, neither rs483352838-v2 nor rs483352838-v3 cannot obtain stable results. Therefore, the two primers are amplified and extended in two reaction wells, respectively, and detected, detection results of the two wells for the same site are combined to judge typing of the samples, and finally stable results enabling simultaneous detection of wild-type samples and mutant samples of the rs483352838 site can be obtained.

Although the present invention is disclosed above, the present invention is not limited thereto. Any person skilled in the art can make various changes and amendments without departing from the spirit and scope of the present invention. Therefore, the protection scope of the present invention shall be based on the scope defined by the claims.

1. A method for genotyping blood group by mass spectrometry detection, including the following steps:
(1) using amplification primers to multiplex PCR amplify an DNA from a blood sample, wherein the amplification primers include forward primers and reverse primers;
(2) purifying an amplification product obtained in Step (1) by an alkaline phosphatase;
(3) using extension primers to extend the purified product in Step (2) by a single base; and
(4) cconducting sample application on a single-base extended product obtained in Step (3) onto a chip for mass spectrometry detection;
wherein the amplification primers and the extension primer mix are included in a tube, and wherein the amplification primers and the extension primers are as below:

| Forward primers | Reverse primers | Extension primers |
|---|---|---|
| SEQ ID NO: 1 | SEQ ID NO: 2 | SEQ ID NO: 3 |
| SEQ ID NO: 4 | SEQ ID NO: 5 | SEQ ID NO: 6 |
| SEQ ID NO: 7 | SEQ ID NO: 8 | SEQ ID NO: 9 |
| SEQ ID NO: 10 | SEQ ID NO: 11 | SEQ ID NO: 12 |
| SEQ ID NO: 13 | SEQ ID NO: 14 | SEQ ID NO: 15 |
| SEQ ID NO: 16 | SEQ ID NO: 17 | SEQ ID NO: 18 |
| SEQ ID NO: 19 | SEQ ID NO: 20 | SEQ ID NO: 21 |
| SEQ ID NO: 22 | SEQ ID NO: 23 | SEQ ID NO: 24 |
| SEQ ID NO: 25 | SEQ ID NO: 26 | SEQ ID NO: 27 |
| SEQ ID NO: 28 | SEQ ID NO: 29 | SEQ ID NO: 30 |
| SEQ ID NO: 31 | SEQ ID NO: 32 | SEQ ID NO: 33 |
| SEQ ID NO: 34 | SEQ ID NO: 35 | SEQ ID NO: 36 |
| SEQ ID NO: 37 | SEQ ID NO: 38 | SEQ ID NO: 39 |
| SEQ ID NO: 40 | SEQ ID NO: 41 | SEQ ID NO: 42 |
| SEQ ID NO: 43 | SEQ ID NO: 44 | SEQ ID NO: 45 |
| SEQ ID NO: 46 | SEQ ID NO: 47 | SEQ ID NO: 48 |
| SEQ ID NO: 49 | SEQ ID NO: 50 | SEQ ID NO: 51 |
| SEQ ID NO: 52 | SEQ ID NO: 53 | SEQ ID NO: 54 |
| SEQ ID NO: 55 | SEQ ID NO: 56 | SEQ ID NO: 57 |
| SEQ ID NO: 58 | SEQ ID NO: 59 | SEQ ID NO: 60 |
| SEQ ID NO: 61 | SEQ ID NO: 62 | SEQ ID NO: 63 |
| SEQ ID NO: 64 | SEQ ID NO: 65 | SEQ ID NO: 66 |
| SEQ ID NO: 67 | SEQ ID NO: 68 | SEQ ID NO: 69 |
| SEQ ID NO: 70 | SEQ ID NO: 71 | SEQ ID NO: 72 |
| SEQ ID NO: 73 | SEQ ID NO: 74 | SEQ ID NO: 75 |
| SEQ ID NO: 76 | SEQ ID NO: 77 | SEQ ID NO: 78 |
| SEQ ID NO: 79 | SEQ ID NO: 80 | SEQ ID NO: 81 |
| SEQ ID NO: 82 | SEQ ID NO: 83 | SEQ ID NO: 84 |
| SEQ ID NO: 85 | SEQ ID NO: 86 | SEQ ID NO: 87 |
| SEQ ID NO: 88 | SEQ ID NO: 89 | SEQ ID NO: 90 |
| SEQ ID NO: 91 | SEQ ID NO: 92 | SEQ ID NO: 93 |
| SEQ ID NO: 94 | SEQ ID NO: 95 | SEQ ID NO: 96 |
| SEQ ID NO: 97 | SEQ ID NO: 98 | SEQ ID NO: 99 |
| SEQ ID NO: 100 | SEQ ID NO: 101 | SEQ ID NO: 102 |
| SEQ ID NO: 103 | SEQ ID NO: 104 | SEQ ID NO: 105 |
| SEQ ID NO: 106 | SEQ ID NO: 107 | SEQ ID NO: 108 |
| SEQ ID NO: 109 | SEQ ID NO: 110 | SEQ ID NO: 111 |
| SEQ ID NO: 112 | SEQ ID NO: 113 | SEQ ID NO: 114 |
| SEQ ID NO: 115 | SEQ ID NO: 116 | SEQ ID NO: 117 |
| SEQ ID NO: 118 | SEQ ID NO: 119 | SEQ ID NO: 120 |
| SEQ ID NO: 121 | SEQ ID NO: 122 | SEQ ID NO: 123 |
| SEQ ID NO: 124 | SEQ ID NO: 125 | SEQ ID NO: 126 |
| SEQ ID NO: 127 | SEQ ID NO: 128 | SEQ ID NO: 129 |
| SEQ ID NO: 130 | SEQ ID NO: 131 | SEQ ID NO: 132 |
| SEQ ID NO: 133 | SEQ ID NO: 134 | SEQ ID NO: 135 |
| SEQ ID NO: 136 | SEQ ID NO: 137 | SEQ ID NO: 138 |
| SEQ ID NO: 139 | SEQ ID NO: 140 | SEQ ID NO: 141 |
| SEQ ID NO: 142 | SEQ ID NO: 143 | SEQ ID NO: 144 |
| SEQ ID NO: 145 | SEQ ID NO: 146 | SEQ ID NO: 147 |
| SEQ ID NO: 148 | SEQ ID NO: 149 | SEQ ID NO: 150 |
| SEQ ID NO: 151 | SEQ ID NO: 152 | SEQ ID NO: 153 |
| SEQ ID NO: 154 | SEQ ID NO: 155 | SEQ ID NO: 156 |
| SEQ ID NO: 157 | SEQ ID NO: 158 | SEQ ID NO: 159 |
| SEQ ID NO: 160 | SEQ ID NO: 161 | SEQ ID NO: 162 |
| SEQ ID NO: 163 | SEQ ID NO: 164 | SEQ ID NO: 165 |
| SEQ ID NO: 166 | SEQ ID NO: 167 | SEQ ID NO: 168 |
| SEQ ID NO: 169 | SEQ ID NO: 170 | SEQ ID NO: 171 |
| SEQ ID NO: 172 | SEQ ID NO: 173 | SEQ ID NO: 174 |
| SEQ ID NO: 175 | SEQ ID NO: 176 | SEQ ID NO: 177 |
| SEQ ID NO: 178 | SEQ ID NO: 179 | SEQ ID NO: 180 |
| SEQ ID NO: 181 | SEQ ID NO: 182 | SEQ ID NO: 183 |
| SEQ ID NO: 184 | SEQ ID NO: 185 | SEQ ID NO: 186 |

2. The method according to claim 1, wherein a concentration of the amplification primers is 0.04 to 0.4 μM in a final concentration.

3. The method according to claim 1, wherein in the Step (1), an amplification reaction system used as below:

| Components | Volume (μL) |
|---|---|
| Water, HPLC grade | 0.8 |
| 10 × PCR Buffer with 20 mM | 0.5 |
| MgCl₂ | |
| 25 mM MgCl₂ | 0.4 |
| 25 mM dNTP Mix | 0.1 |
| 0.2-2.0 uM Primer Mix | 1 |
| 5 U/μl PCR Enzyme | 0.2 |
| 5-20 ng/μL DNA | 2 |
| Total volume | 5 |

4. The method according to claim 3, wherein in the Step (1), cycle conditions of PCR amplification reaction are as follows: 95° C., 2 minutes; 45 cycles: 95° C., 30 seconds, 56° C., 30 seconds, 72° C., 60 seconds, 72° C., 5 minutes; keeping a temperature of 4° C.

5. The method according to claim 4, wherein the alkaline phosphatase in Step (2) is a shrimp alkaline phosphatase, and a premixed solution system for purification treatment with the alkaline phosphatase in Step (2) as below:

| Components | Volume (μL) |
|---|---|
| Nanopure Water, Autoclaved | 1.53 |
| SAP Buffer | 0.17 |
| SAP Enzyme (1.7 U/ul) | 0.30 |
| Total volume | 2 |

6. The method according to claim 5, wherein a single-base extension and amplification system in Step (3) is below:

| Components | Volume (μL) |
|---|---|
| Nanopure Water, Autoclaved | 0.619 |
| iPLEX Buffer | 0.200 |
| iPLEX Termination Mix | 0.200 |
| Extend Primer Mix | 0.94 |
| iPLEX Enzyme | 0.041 |
| Total volume | 2 |


SEQUENCE LISTING (186 sequences)

SEQ ID NO: 1 - DNA, 30 bp, synthetic construct
  source 1..30: mol_type = other DNA
acgttggatgattgggatgtttccagccgt

SEQ ID NO: 2 - DNA, 30 bp, synthetic construct
  source 1..30: mol_type = other DNA
acgttggatgtggaaacctgtggcatcctc

SEQ ID NO: 3 - DNA, 18 bp, synthetic construct
  source 1..18: mol_type = other DNA
aaggtgctgctgcctgcg

SEQ ID NO: 4 - DNA, 30 bp, synthetic construct
  source 1..30: mol_type = other DNA
acgttggatgagccatgaagaagatgaacc

SEQ ID NO: 5 - DNA, 30 bp, synthetic construct
  source 1..30: mol_type = other DNA
acgttggatgtgctgtgcaacattaagccc

SEQ ID NO: 6 - DNA, 17 bp, synthetic construct
  source 1..17: mol_type = other DNA
ccctgactgtggtcttc

SEQ ID NO: 7 - DNA, 30 bp, synthetic construct
  source 1..30: mol_type = other DNA
acgttggatgtgttctcggttttccctggc

SEQ ID NO: 8 - DNA, 30 bp, synthetic construct
  source 1..30: mol_type = other DNA
acgttggatgaagagcctgaggaggcctat

SEQ ID NO: 9 - DNA, 15 bp, synthetic construct
  source 1..15: mol_type = other DNA
gttcctgcccccagc

SEQ ID NO: 10 - DNA, 31 bp, synthetic construct
  source 1..31: mol_type = other DNA
acgttggatgaggccagcacagccagccttg

SEQ ID NO: 11 - DNA, 30 bp, synthetic construct
  source 1..30: mol_type = other DNA
acgttggatgatttgctcctgtgaccactg

SEQ ID NO: 12 - DNA, 26 bp, synthetic construct
  source 1..26: mol_type = other DNA
tatgtccggcgctgcctgcccctctg

SEQ ID NO: 13 - DNA, 30 bp, synthetic construct
  source 1..30: mol_type = other DNA
acgttggatgccctcttcttgtggatgttc

SEQ ID NO: 14 - DNA, 30 bp, synthetic construct
  source 1..30: mol_type = other DNA
acgttggatgacatggcattcttcctttgg

SEQ ID NO: 15 - DNA, 17 bp, synthetic construct
  source 1..17: mol_type = other DNA
aggacttctcagcagag

SEQ ID NO: 16 - DNA, 30 bp, synthetic construct
  source 1..30: mol_type = other DNA
acgttggatgtgacttagggatgaaggctc

SEQ ID NO: 17 - DNA, 30 bp, synthetic construct
  source 1..30: mol_type = other DNA
acgttggatgtggtgggacatccttatcag

SEQ ID NO: 18 - DNA, 17 bp, synthetic construct
  source 1..17: mol_type = other DNA
tggtgactccatttctg

SEQ ID NO: 19 - DNA, 30 bp, synthetic construct
  source 1..30: mol_type = other DNA
acgttggatggctttggtaatgagacacgc

SEQ ID NO: 20 - DNA, 30 bp, synthetic construct; identical to SEQ ID NO: 19
  source 1..30: mol_type = other DNA

SEQ ID NO: 21 - DNA, 21 bp, synthetic construct
  source 1..21: mol_type = other DNA
caatcagatgaacaattgacg

SEQ ID NO: 22 - DNA, 30 bp, synthetic construct
  source 1..30: mol_type = other DNA
acgttggatgccagcgagttcaagaagaag

SEQ ID NO: 23 - DNA, 30 bp, synthetic construct
  source 1..30: mol_type = other DNA
acgttggatgcagaaccgatgctgatgaag

SEQ ID NO: 24 - DNA, 20 bp, synthetic construct
  source 1..20: mol_type = other DNA
caatgctgatgaagacaaag

SEQ ID NO: 25 - DNA, 30 bp, synthetic construct
  source 1..30: mol_type = other DNA
acgttggatgcaaaccatctctgaggacac

SEQ ID NO: 26 - DNA, 30 bp, synthetic construct
  source 1..30: mol_type = other DNA
acgttggatggcttgactcacttggttctg

SEQ ID NO: 27 - DNA, 17 bp, synthetic construct
  source 1..17: mol_type = other DNA
tcgaggggtgggacaag

SEQ ID NO: 28 - DNA, 29 bp, synthetic construct
  source 1..29: mol_type = other DNA
acgttggatgtgatgtggcccacactctg

SEQ ID NO: 29 - DNA, 30 bp, synthetic construct
  source 1..30: mol_type = other DNA
acgttggatgaatacccggtggggaacaac

SEQ ID NO: 30 - DNA, 15 bp, synthetic construct
  source 1..15: mol_type = other DNA
ggaacaaccagacgg

SEQ ID NO: 31 - DNA, 30 bp, synthetic construct
  source 1..30: mol_type = other DNA
acgttggatgaaattcctggcgagaaggac

SEQ ID NO: 32 - DNA, 30 bp, synthetic construct
  source 1..30: mol_type = other DNA
acgttggatgtgaagaacttacgattgcag

SEQ ID NO: 33 - DNA, 20 bp, synthetic construct
  source 1..20: mol_type = other DNA
tgcagaactcttcaatatct

SEQ ID NO: 34 - DNA, 30 bp, synthetic construct; identical to SEQ ID NO: 32
  source 1..30: mol_type = other DNA

SEQ ID NO: 35 - DNA, 30 bp, synthetic construct; identical to SEQ ID NO: 31
  source 1..30: mol_type = other DNA

SEQ ID NO: 36 - DNA, 20 bp, synthetic construct
  source 1..20: mol_type = other DNA
cgccttaagggcagtcaatg

SEQ ID NO: 37 - DNA, 30 bp, synthetic construct
  source 1..30: mol_type = other DNA
acgttggatgagaaatcatgccctaatccg

SEQ ID NO: 38 - DNA, 30 bp, synthetic construct
  source 1..30: mol_type = other DNA
acgttggatgtgagaaggagatggttgcac

SEQ ID NO: 39 - DNA, 21 bp, synthetic construct
  source 1..21: mol_type = other DNA
ctacatcaatctgaccatttc

SEQ ID NO: 40 - DNA, 30 bp, synthetic construct
  source 1..30: mol_type = other DNA
acgttggatgtttaaaagatgttggaattg

SEQ ID NO: 41 - DNA, 30 bp, synthetic construct
  source 1..30: mol_type = other DNA
acgttggatgtaatggtcacgttccccttg

SEQ ID NO: 42 - DNA, 20 bp, synthetic construct
  source 1..20: mol_type = other DNA
ggtgtcaatttgtggtggtg

SEQ ID NO: 43 - DNA, 30 bp, synthetic construct
  source 1..30: mol_type = other DNA
acgttggatgacgttcctgaagatgagcgg

SEQ ID NO: 44 - DNA, 30 bp, synthetic construct
  source 1..30: mol_type = other DNA
acgttggatgagtgctgtgggtggtgaagt

SEQ ID NO: 45 - DNA, 21 bp, synthetic construct
  source 1..21: mol_type = other DNA
aggtggtggtgaagtccacgc

SEQ ID NO: 46 - DNA, 30 bp, synthetic construct
  source 1..30: mol_type = other DNA
acgttggatgagagtcatccagcaggttac

SEQ ID NO: 47 - DNA, 30 bp, synthetic construct
  source 1..30: mol_type = other DNA
acgttggatgtgattccttcccagatggag

SEQ ID NO: 48 - DNA, 18 bp, synthetic construct
  source 1..18: mol_type = other DNA
gcccagatggagactatg

SEQ ID NO: 49 - DNA, 30 bp, synthetic construct
  source 1..30: mol_type = other DNA
acgttggatgctgacctcagattcttgtcc

SEQ ID NO: 50 - DNA, 30 bp, synthetic construct
  source 1..30: mol_type = other DNA
acgttggatgacaatgtccattatggtggg

SEQ ID NO: 51 - DNA, 17 bp, synthetic construct
  source 1..17: mol_type = other DNA
ctccattctgccatccg

SEQ ID NO: 52 - DNA, 30 bp, synthetic construct
  source 1..30: mol_type = other DNA
acgttggatgtctgaagtcacggcagtttc

SEQ ID NO: 53 - DNA, 29 bp, synthetic construct
  source 1..29: mol_type = other DNA
acgttggatgtctgcctccaccacaatgc

SEQ ID NO: 54 - DNA, 23 bp, synthetic construct
  source 1..23: mol_type = other DNA
ttcccccaccacaatgcatacta

SEQ ID NO: 55 - DNA, 30 bp, synthetic construct
  source 1..30: mol_type = other DNA
acgttggatgatgcatcctggactggaaac

SEQ ID NO: 56 - DNA, 30 bp, synthetic construct
  source 1..30: mol_type = other DNA
acgttggatgtagaagcatggaacgagagc

SEQ ID NO: 57 - DNA, 24 bp, synthetic construct
  source 1..24: mol_type = other DNA
cttttgtctgtcttccatgtcact

SEQ ID NO: 58 - DNA, 30 bp, synthetic construct
  source 1..30: mol_type = other DNA
acgttggatggcttggtgaattaaccagcc

SEQ ID NO: 59 - DNA, 30 bp, synthetic construct
  source 1..30: mol_type = other DNA
acgttggatgttcttttgcaggccactatg

SEQ ID NO: 60 - DNA, 25 bp, synthetic construct
  source 1..25: mol_type = other DNA
tatgtacatggtatttgtatctatg

SEQ ID NO: 61 - DNA, 29 bp, synthetic construct
  source 1..29: mol_type = other DNA
acgttggatgctgcctcctgaccatgcaa

SEQ ID NO: 62 - DNA, 30 bp, synthetic construct
  source 1..30: mol_type = other DNA
acgttggatggatgtggaggtgaagttttc

SEQ ID NO: 63 - DNA, 23 bp, synthetic construct
  source 1..23: mol_type = other DNA
gctcttggtggacatatttagtt

SEQ ID NO: 64 - DNA, 30 bp, synthetic construct; identical to SEQ ID NO: 56
  source 1..30: mol_type = other DNA

SEQ ID NO: 65 - DNA, 30 bp, synthetic construct; identical to SEQ ID NO: 55
  source 1..30: mol_type = other DNA

SEQ ID NO: 66 - DNA, 25 bp, synthetic construct
  source 1..25: mol_type = other DNA
ctagtagcctcaccgtggcagcctc

SEQ ID NO: 67 - DNA, 30 bp, synthetic construct
  source 1..30: mol_type = other DNA
acgttggatggacataggccaaagggaatg

SEQ ID NO: 68 - DNA, 30 bp, synthetic construct
  source 1..30: mol_type = other DNA
acgttggatgtgtcccttgcaaggattacc

SEQ ID NO: 69 - DNA, 19 bp, synthetic construct
  source 1..19: mol_type = other DNA
cggattacctgacccagaa

SEQ ID NO: 70 - DNA, 30 bp, synthetic construct
  source 1..30: mol_type = other DNA
acgttggatgtgagcttaatacctaccccc

SEQ ID NO: 71 - DNA, 30 bp, synthetic construct
  source 1..30: mol_type = other DNA
acgttggatggggttgtatcgcagtttcac

SEQ ID NO: 72 - DNA, 18 bp, synthetic construct
  source 1..18: mol_type = other DNA
tgattgagggttctttcg

SEQ ID NO: 73 - DNA, 30 bp, synthetic construct
  source 1..30: mol_type = other DNA
acgttggatggcaaggtgctattgaaagcc

SEQ ID NO: 74 - DNA, 30 bp, synthetic construct
  source 1..30: mol_type = other DNA
acgttggatgacgtggagaaaaatggtcgc

SEQ ID NO: 75 - DNA, 23 bp, synthetic construct
  source 1..23: mol_type = other DNA
aagtggtcgctacagcatctctc

SEQ ID NO: 76 - DNA, 30 bp, synthetic construct
  source 1..30: mol_type = other DNA
acgttggatgcatctactttggactctggg

SEQ ID NO: 77 - DNA, 30 bp, synthetic construct
  source 1..30: mol_type = other DNA
acgttggatgaagagccaggaggtgggttt

SEQ ID NO: 78 - DNA, 16 bp, synthetic construct
  source 1..16: mol_type = other DNA
gagcgccatgaacatt

SEQ ID NO: 79 - DNA, 30 bp, synthetic construct
  source 1..30: mol_type = other DNA
acgttggatgaaatgtccgtgctgtgtctc

SEQ ID NO: 80 - DNA, 30 bp, synthetic construct
  source 1..30: mol_type = other DNA
acgttggatgtggcattgtagccatagagc

SEQ ID NO: 81 - DNA, 19 bp, synthetic construct
  source 1..19: mol_type = other DNA
cccagatgctattaatgac

SEQ ID NO: 82 - DNA, 30 bp, synthetic construct
  source 1..30: mol_type = other DNA
acgttggatgctcctgtcttaacaggactc

SEQ ID NO: 83 - DNA, 30 bp, synthetic construct
  source 1..30: mol_type = other DNA
acgttggatgcagagagctgttgaaacccc

SEQ ID NO: 84 - DNA, 20 bp, synthetic construct
  source 1..20: mol_type = other DNA
ccagagtccaaagtagatgt

SEQ ID NO: 85 - DNA, 29 bp, synthetic construct
  source 1..29: mol_type = other DNA
acgttggatgtgacttagtattgtctcac

SEQ ID NO: 86 - DNA, 30 bp, synthetic construct
  source 1..30: mol_type = other DNA
acgttggatgatatcgaggctgatgaatgg

SEQ ID NO: 87 - DNA, 20 bp, synthetic construct
  source 1..20: mol_type = other DNA
aatggagaagatgattgttc

SEQ ID NO: 88 - DNA, 30 bp, synthetic construct
  source 1..30: mol_type = other DNA
acgttggatgcacaaccattgcatcttggc

SEQ ID NO: 89 - DNA, 30 bp, synthetic construct
  source 1..30: mol_type = other DNA
acgttggatgcagacaaggaagacataccg

SEQ ID NO: 90 - DNA, 22 bp, synthetic construct
  source 1..22: mol_type = other DNA
agacataccgtaaatccatatc

SEQ ID NO: 91 - DNA, 30 bp, synthetic construct
  source 1..30: mol_type = other DNA
acgttggatgcgacctgccaatttcaaatg

SEQ ID NO: 92 - DNA, 30 bp, synthetic construct
  source 1..30: mol_type = other DNA
acgttggatgatcagccaaagcacttaccc

SEQ ID NO: 93 - DNA, 22 bp, synthetic construct
  source 1..22: mol_type = other DNA
gcaaacccactaatacttactt

SEQ ID NO: 94 - DNA, 30 bp, synthetic construct
  source 1..30: mol_type = other DNA
acgttggatgggaaatggccatactgactc

SEQ ID NO: 95 - DNA, 30 bp, synthetic construct
  source 1..30: mol_type = other DNA
acgttggatgcgcatctctggtaaatggac

SEQ ID NO: 96 - DNA, 20 bp, synthetic construct
  source 1..20: mol_type = other DNA
ttccttaaactttaaccgaa

SEQ ID NO: 97 - DNA, 30 bp, synthetic construct
  source 1..30: mol_type = other DNA
acgttggatggaacggtggtaacttaccag

SEQ ID NO: 98 - DNA, 30 bp, synthetic construct
  source 1..30: mol_type = other DNA
acgttggatgctggtgcaatatattgaccg

SEQ ID NO: 99 - DNA, 15 bp, synthetic construct
  source 1..15: mol_type = other DNA
ttgaccgttctccca

SEQ ID NO: 100 - DNA, 30 bp, synthetic construct
  source 1..30: mol_type = other DNA
acgttggatgagggctcagattacctgtag

SEQ ID NO: 101 - DNA, 30 bp, synthetic construct
  source 1..30: mol_type = other DNA
acgttggatgtccttgattgccacaggttg

SEQ ID NO: 102 - DNA, 22 bp, synthetic construct
  source 1..22: mol_type = other DNA
gagagggcacctacattatcca

SEQ ID NO: 103 - DNA, 30 bp, synthetic construct
  source 1..30: mol_type = other DNA
acgttggatgtgtggctgcttgtcgtggag

SEQ ID NO: 104 - DNA, 29 bp, synthetic construct
  source 1..29: mol_type = other DNA
cgttggatgctgaacttgatccagatgcc

SEQ ID NO: 105 - DNA, 19 bp, synthetic construct
  source 1..19: mol_type = other DNA
tcgatgcccattgccagac

SEQ ID NO: 106 - DNA, 30 bp, synthetic construct
  source 1..30: mol_type = other DNA
acgttggatgcaagttctcagctgcaaacg

SEQ ID NO: 107 - DNA, 30 bp, synthetic construct
  source 1..30: mol_type = other DNA
acgttggatgtctggatcaagttcaggcac

SEQ ID NO: 108 - DNA, 16 bp, synthetic construct
  source 1..16: mol_type = other DNA
ctggtctcctgctcct

SEQ ID NO: 109 - DNA, 29 bp, synthetic construct
  source 1..29: mol_type = other DNA
acgttggatgtacaccggttgcagcgcca

SEQ ID NO: 110 - DNA, 29 bp, synthetic construct
  source 1..29: mol_type = other DNA
acgttggatgggctcccgacgccttcgtg

SEQ ID NO: 111 - DNA, 25 bp, synthetic construct
  source 1..25: mol_type = other DNA
ggctccagccccggcccccgagccc

SEQ ID NO: 112 - DNA, 29 bp, synthetic construct
  source 1..29: mol_type = other DNA
acgttggatgaaggcgctggcgctgcaac

SEQ ID NO: 113 - DNA, 29 bp, synthetic construct
  source 1..29: mol_type = other DNA
acgttggatgaggcactgaaagcccggtc

SEQ ID NO: 114 - DNA, 26 bp, synthetic construct
  source 1..26: mol_type = other DNA
ttattgagccggcgccgggcgccggg

SEQ ID NO: 115 - DNA, 29 bp, synthetic construct
  source 1..29: mol_type = other DNA
acgttggatgaaggcgctggcgctgcaac

SEQ ID NO: 116 - DNA, 28 bp, synthetic construct
  source 1..28: mol_type = other DNA
cgttggatgaggcactgaaagcccggtc

SEQ ID NO: 117 - DNA, 22 bp, synthetic construct
  source 1..22: mol_type = other DNA
gcccgaggaggcggcgccgggc

SEQ ID NO: 118 - DNA, 29 bp, synthetic construct
  source 1..29: mol_type = other DNA
acgttggatgacagaccgaccgctcggga

SEQ ID NO: 119 - DNA, 30 bp, synthetic construct
  source 1..30: mol_type = other DNA
acgttggatggtgtcgtgcattgtgacctg

SEQ ID NO: 120 - DNA, 19 bp, synthetic construct
  source 1..19: mol_type = other DNA
gatatcagccgaggctagg

SEQ ID NO: 121 - DNA, 30 bp, synthetic construct
  source 1..30: mol_type = other DNA
acgttggatgcaaagtacttcttccagtcc

SEQ ID NO: 122 - DNA, 30 bp, synthetic construct
  source 1..30: mol_type = other DNA
acgttggatgaagaagtggtgttccatccg

SEQ ID NO: 123 - DNA, 16 bp, synthetic construct
  source 1..16: mol_type = other DNA
tcaccaccctgccccc

SEQ ID NO: 124 - DNA, 30 bp, synthetic construct
  source 1..30: mol_type = other DNA
acgttggatgtacattttcatggcctcgtg

SEQ ID NO: 125 - DNA, 30 bp, synthetic construct
  source 1..30: mol_type = other DNA
acgttggatgtccacgtgtggaacaagaag

SEQ ID NO: 126 - DNA, 19 bp, synthetic construct
  source 1..19: mol_type = other DNA
tcatgcccgctactgcccc

SEQ ID NO: 127 - DNA, 29 bp, synthetic construct
  source 1..29: mol_type = other DNA
acgttggatgtgcacggccgcgtaccagt

SEQ ID NO: 128 - DNA, 30 bp, synthetic construct
  source 1..30: mol_type = other DNA
acgttggatgtcacttctgagctgcttccc

SEQ ID NO: 129 - DNA, 21 bp, synthetic construct
  source 1..21: mol_type = other DNA
ctgagctgcttcccgaatgtc

SEQ ID NO: 130 - DNA, 30 bp, synthetic construct; identical to SEQ ID NO: 128
  source 1..30: mol_type = other DNA

SEQ ID NO: 131 - DNA, 29 bp, synthetic construct
  source 1..29: mol_type = other DNA
acgttggatgtgcacggccgcgtaccagt

SEQ ID NO: 132 - DNA, 16 bp, synthetic construct
  source 1..16: mol_type = other DNA
gtcggccaggggtgtg

SEQ ID NO: 133 - DNA, 30 bp, synthetic construct
  source 1..30: mol_type = other DNA
acgttggatgagaagtgagatgcccaggtg

SEQ ID NO: 134 - DNA, 30 bp, synthetic construct
  source 1..30: mol_type = other DNA
acgttggatgacttcctgttcatgtgctcg

SEQ ID NO: 135 - DNA, 15 bp, synthetic construct
  source 1..15: mol_type = other DNA
tgctcggtggagtcg

SEQ ID NO: 136 - DNA, 30 bp, synthetic construct; identical to SEQ ID NO: 133
  source 1..30: mol_type = other DNA

SEQ ID NO: 137 - DNA, 30 bp, synthetic construct; identical to SEQ ID NO: 134
  source 1..30: mol_type = other DNA

SEQ ID NO: 138 - DNA, 22 bp, synthetic construct
  source 1..22: mol_type = other DNA
atcccacgtgctggtcctgatg

SEQ ID NO: 139 - DNA, 30 bp, synthetic construct; identical to SEQ ID NO: 124
  source 1..30: mol_type = other DNA

SEQ ID NO: 140 - DNA, 30 bp, synthetic construct; identical to SEQ ID NO: 125
  source 1..30: mol_type = other DNA

SEQ ID NO: 141 - DNA, 23 bp, synthetic construct
  source 1..23: mol_type = other DNA
tggaacaagaagagccagggcac

SEQ ID NO: 142 - DNA, 30 bp, synthetic construct
  source 1..30: mol_type = other DNA
acgttggatgggtcataatggtgtggttgc

SEQ ID NO: 143 - DNA, 30 bp, synthetic construct
  source 1..30: mol_type = other DNA
acgttggatgtgtgacgtttgctggcgatg

SEQ ID NO: 144 - DNA, 15 bp, synthetic construct
  source 1..15: mol_type = other DNA
caccgtggaaagact

SEQ ID NO: 145 - DNA, 30 bp, synthetic construct
  source 1..30: mol_type = other DNA
acgttggatgtcttcccgaaggtggtcgtg

SEQ ID NO: 146 - DNA, 30 bp, synthetic construct
  source 1..30: mol_type = other DNA
acgttggatgactttcttccaccatctccg

SEQ ID NO: 147 - DNA, 18 bp, synthetic construct
  source 1..18: mol_type = other DNA
gggaacagatccgcagag

SEQ ID NO: 148 - DNA, 29 bp, synthetic construct
  source 1..29: mol_type = other DNA
acgttggatgaagaggcgcagagtgtgct

SEQ ID NO: 149 - DNA, 30 bp, synthetic construct
  source 1..30: mol_type = other DNA
acgttggatggcataacctgcagatagtcc

SEQ ID NO: 150 - DNA, 19 bp, synthetic construct
  source 1..19: mol_type = other DNA
ttccgatagtccccacggc

SEQ ID NO: 151 - DNA, 31 bp, synthetic construct
  source 1..31: mol_type = other DNA
acgttggatgtgattaagaaaaggaaacccg

SEQ ID NO: 152 - DNA, 31 bp, synthetic construct
  source 1..31: mol_type = other DNA
acgttggatgggcttggcctcccaaaattat

SEQ ID NO: 153 - DNA, 15 bp, synthetic construct
  source 1..15: mol_type = other DNA
tggacaagttgtccc

SEQ ID NO: 154 - DNA, 30 bp, synthetic construct
  source 1..30: mol_type = other DNA
acgttggatgttgatctccccaccgagaag

SEQ ID NO: 155 - DNA, 30 bp, synthetic construct
  source 1..30: mol_type = other DNA
acgttggatgagcgtgaggcctcttctgtg

SEQ ID NO: 156 - DNA, 16 bp, synthetic construct
  source 1..16: mol_type = other DNA
gctgctgccgtcctcc

SEQ ID NO: 157 - DNA, 30 bp, synthetic construct; identical to SEQ ID NO: 155
  source 1..30: mol_type = other DNA

SEQ ID NO: 158 - DNA, 29 bp, synthetic construct
  source 1..29: mol_type = other DNA
acgttggatgacagcatgcagccccagga

SEQ ID NO: 159 - DNA, 24 bp, synthetic construct
  source 1..24: mol_type = other DNA
atcatgcagccccaggagagccac

SEQ ID NO: 160 - DNA, 30 bp, synthetic construct; identical to SEQ ID NO: 155
  source 1..30: mol_type = other DNA

SEQ ID NO: 161 - DNA, 30 bp, synthetic construct; identical to SEQ ID NO: 154
  source 1..30: mol_type = other DNA

SEQ ID NO: 162 - DNA, 19 bp, synthetic construct
  source 1..19: mol_type = other DNA
aggcagcagggacggagtc

SEQ ID NO: 163 - DNA, 29 bp, synthetic construct
  source 1..29: mol_type = other DNA
acgttggatggagacaccagcctgctatg

SEQ ID NO: 164 - DNA, 30 bp, synthetic construct
  source 1..30: mol_type = other DNA
acgttggatgctgtgtccagcacagaagag

SEQ ID NO: 165 - DNA, 25 bp, synthetic construct
  source 1..25: mol_type = other DNA
cagcacagaagaggcctcacgctgc

SEQ ID NO: 166 - DNA, 30 bp, synthetic construct
  source 1..30: mol_type = other DNA
acgttggatgcacctccagttggttctcac

SEQ ID NO: 167 - DNA, 30 bp, synthetic construct
  source 1..30: mol_type = other DNA
acgttggatgaagatgatccagaagagggc

SEQ ID NO: 168 - DNA, 25 bp, synthetic construct
  source 1..25: mol_type = other DNA
caaggccacgccgcccagcaccttc

SEQ ID NO: 169 - DNA, 30 bp, synthetic construct; identical to SEQ ID NO: 155
  source 1..30: mol_type = other DNA

SEQ ID NO: 170 - DNA, 30 bp, synthetic construct; identical to SEQ ID NO: 154
  source 1..30: mol_type = other DNA

SEQ ID NO: 171 - DNA, 18 bp, synthetic construct
  source 1..18: mol_type = other DNA
catcccgctgcagcccca

SEQ ID NO: 172 - DNA, 31 bp, synthetic construct
  source 1..31: mol_type = other DNA
acgttggatgggtgtaacccctctcttctcc

SEQ ID NO: 173 - DNA, 30 bp, synthetic construct
  source 1..30: mol_type = other DNA
acgttggatgcggtccgaccactcattaga

SEQ ID NO: 174 - DNA, 24 bp, synthetic construct
  source 1..24: mol_type = other DNA
ctactcctccaccagcttctcctc

SEQ ID NO: 175 - DNA, 30 bp, synthetic construct
  source 1..30: mol_type = other DNA
acgttggatgatgcagagctgctggtgacg

SEQ ID NO: 176 - DNA, 30 bp, synthetic construct
  source 1..30: mol_type = other DNA
acgttggatgatgcccaggaaagcagagac

SEQ ID NO: 177 - DNA, 20 bp, synthetic construct
  source 1..20: mol_type = other DNA
aataagagacagggcccccg

SEQ ID NO: 178 - DNA, 29 bp, synthetic construct
  source 1..29: mol_type = other DNA
acgttggatgagcctccccatgggtgaag

SEQ ID NO: 179 - DNA, 30 bp, synthetic construct
  source 1..30: mol_type = other DNA
acgttggatgcaggtgtaacccctctcttc

SEQ ID NO: 180 - DNA, 15 bp, synthetic construct
  source 1..15: mol_type = other DNA
ctcggaggctcccag

SEQ ID NO: 181 - DNA, 30 bp, synthetic construct
  source 1..30: mol_type = other DNA
acgttggatggaagagaggggttacacctg

SEQ ID NO: 182 - DNA, 29 bp, synthetic construct
  source 1..29: mol_type = other DNA
acgttggatgcttctggaaccgcttcctc

SEQ ID NO: 183 - DNA, 19 bp, synthetic construct
  source 1..19: mol_type = other DNA
ggggcgcttcctccccaaa

SEQ ID NO: 184 - DNA, 30 bp, synthetic construct
  source 1..30: mol_type = other DNA
acgttggatgtcttccggttctccttcgtg

SEQ ID NO: 185 - DNA, 30 bp, synthetic construct
  source 1..30: mol_type = other DNA
acgttggatgagctagccactagttacctg

SEQ ID NO: 186 - DNA, 17 bp, synthetic construct
  source 1..17: mol_type = other DNA
agttacctgcaggccgt